(19) United States
(12) Patent Application Publication
(10) Pub. No.: US 2014/0121694 A1
(43) Pub. Date: May 1, 2014

(54) TROCAR DISPENSER AND GRIP RECEIVER SAFETY SYSTEM

(71) Applicants: Paul Lambert, El Dorado Hills, CA (US); Carlos Gutierrez, El Dorado Hills, CA (US)

(72) Inventors: Paul Lambert, El Dorado Hills, CA (US); Carlos Gutierrez, El Dorado Hills, CA (US)

(21) Appl. No.: 13/664,155

(22) Filed: Oct. 30, 2012

(51) Int. Cl. A61B 17/34

(52) U.S. Cl. CPC A61B 17/3496; A61B 17/3415; A61B 17/3417
USPC 606/185

(57) ABSTRACT

A disposable system for safely deploying, retrieving and handling a trocar during open surgery. An embodiment comprises a dispenser handle that houses a trocar within a protective sheath during deployment of the trocar within the surgical wound area; and an adjoining receiver that provides a target, shield and active backstop when retrieving the trocar tip from the surgical site. The dispenser handle is shaped ergonomically for easy manipulation; contains a sheath to safely cover the trocar tip during manipulation; and integrates a coupler that permits independent motion between it and the sheath, while releasably gripping the trocar until deployed within the surgical site. The adjoining receiver is comprised of a compact receiver handle grip; an integrated trocar-locking mechanism that traps the trocar when extracting it, while trailing drainage tubing from the surgical site; and a removable safety shield.

### CROSS-REFERENCES TO RELATED APPLICATIONS

Not Applicable.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

I. Field of Invention

In the field of surgery, this invention relates to a device to safely deploy sharp surgical instruments, such as a needle or trocar, used to penetrate through the patient's skin in applications such as installing a drain, catheter or similar device that extends from within a surgical site to outside the surgical site. This invention implements features that improve safety such as reducing exposure to the patient of accidental injury when inserting the trocar within the surgical site and reducing exposure to the clinician of accidental sharps injuries when extracting the trocar from the surgical site. This invention improves the methods of current wound drain insertion procedures in terms of ergonomics, functionality and maneuverability. This invention implements features to create a simpler and more cost-effective device than other devices that exist in the field.

### Overview of the Current Field

General surgery involving open wound sites may require wound drainage to drain a surgical site of an unwanted accumulation of blood, serous fluid and other bodily fluids. Wound drainage for open surgical procedures is typically initiated through the use of a sharp instrument such as a needle or trocar to create a small puncture wound opening in the tissues adjacent to a surgical site opening. A trocar is typically fashioned as a sharpened, heavy gauge needle or as a hollow shaft with a pointed tip. The trocar is used to puncture the patient's tissue and create a wound opening through which drainage tubing (attached to the distal stem of the trocar) is threaded. The drainage tubing provides a pathway for bodily fluids to exit the site of surgery through the tubing after the wound opening is closed. The tubing is flexible and typically made from plastic, rubber, or similar soft materials. The distal end of the tubing that remains within the surgical site is fashioned to collect the fluids created or collected within the wound by perforations, mesh, foam or other absorbent materials. The fluids exit the surgical site through the tubing until the fluids are collected outside the patient's body within a collection bag or reservoir. The trocar is most often a single use device, so that after a trocar passes through a patient's tissue, the tubing is cut adjacent to the stem of the trocar and the trocar is discarded into a sharps container.

A clinician deploying a trocar must do so with precision and control throughout the process: while preparing the trocar for use, inserting the trocar through the patient's skin, extricating the trocar from the patient's body, handling the trocar when cutting the drainage tubing from the distal end of the trocar, and safely disposing the trocar into a biohazard disposal container designated for sharps.

Modernly, trocars may be delivered by the manufacturer with a safety cover that is removed before usage in surgery. It has been noted that such covers may occasionally stick to the trocar when removing the cover. Therefore, the clinician must use care when using one hand to pull the cover off the trocar so that the other hand (holding the trocar) does not uncontrollably swing in the opposite, reactionary direction possibly resulting in an inadvertent sharps injury.

The tip of the trocar is typically extremely sharp or pointed, and fashioned like the point of a spear, a bullet-like tip, blade edges, or some other penetrating shape. When positioning the trocar tip within the surgical site, the clinician typically uses a gloved hand or a manual tool such as wrench or other device to maneuver the trocar tip inside the surgical site, taking care to avoid accidentally puncturing or injuring the patient's tissues or organs exposed within the open site.

Although the trocar tip is sharp or pointed, penetrating soft but elastic tissues may nevertheless require significant pushing force; therefore the clinician must carefully target the direction of insertion and control his or her pushing force. Backpressure disappears upon successfully penetrating the skin and the trocar may unexpectedly surge forward along the pathway of insertion; therefore the clinician must use controlled pushing pressure to avoid potentially tearing the patient's skin.

Likewise, during and after penetrating the tissues and when extricating the trocar from the exit pathway, the clinician must use care to avoid sharps injuries by the exposed trocar tip. Historically, the clinician uses gloved fingers or a manual tool such as pliers or other tool to grip the emerging trocar tip. Therefore, the clinician must be wary of the unexpected or sudden emergence of the trocar tip through the skin after penetration. Further, when pulling the trocar through the exit pathway in the skin, the clinician must prevent his hand slipping along the shaft to the sharp tip due to blood or other bodily fluids making the trocar shaft slippery.

Before pulling the trocar through the skin of the patient, the clinician may have difficulty finding the protruding trocar tip as it emerges from the surgical area. Moreover, the exposed trocar tip presents a hazard because it may be slippery to handle due to bodily fluids and an inadvertent sharps injury may pose a risk of exposure to bloodborne pathogens. Clinicians use a gloved hand alone or with manual grippers such as pliers or other device to grip the trocar tip and extract it from the surgical site.

The trocar is most often a single use device. After extricating the trocar, the clinician cuts the drainage tubing from the stem of the trocar, places the cut end of the tubing into a reservoir or container to channel the bodily fluids, and discards the trocar (perhaps with a small segment of trailing tubing) into a sharps container. When doing so, the clinician again must carefully use a gloved hand to hold the sharp trocar tip, which remains slippery from any bodily fluids present during the procedure, and avoid sharps injuries.

Trocars have been fashioned with straight or curved shafts. Trocars may contain a bend at roughly midway in the shaft, fashioned with a slightly acute angle measured from the axial direction of the shaft. The bend is intended to provide ergonomic benefit to the clinician by permitting the wrist to remain relatively straight while manipulating the trocar tip, which is forward of the bend, at an angled direction into the patient's skin.

II. Background of Environment

Inherent in the use of sharp surgical instruments such as trocars are risks of sharps injuries to both patients and clinicians. First, the trocar is handled without any patient safeguards which may result in accidental puncture of organs or tissues inside the patient. Second, the clinician is exposed to sharps injuries as the trocar exits the patient's skin, especially given the force required to penetrate the skin, and to risks of contamination by the commensurate bodily fluids involved in these types of procedures.

Safety concerns regarding sharp surgical instruments such as trocars have been a serious issue in recent decades. The US Congress recognized the need for safety improvements via regulatory and legislative measures, such as the Bloodborne Pathogen Standard (“BPS”) (29 CFR 1910.1030) promulgated by the Occupational Safety and Health Administration (“OSHA”) in 1991. The BPS stated employer requirements for planning and procedures to protect health-related workers from exposure to bloodborne pathogens, such as: establishing and maintaining an exposure control plan, implementing universal precautions and engineering controls and work practice controls, keeping certain records, providing personal protective equipment, making available hepatitis B vaccinations and post-exposure follow-ups, providing information and training, and using hazards labels and signs.

On Nov. 6, 2000, the Needlestick Safety and Prevention Act (“NSPA”) (Pub. L. 106-430) was signed into law and effected a revision of the BPS in 2001. In the NSPA, Congress directed OSHA to enhance the BPS by requiring employers to: select and implement medical sharps devices that incorporated safety protections, maintain a detailed sharps injury log, and provide for greater employee involvement in evaluating and selecting safety devices and personal protective equipment.

Several of the US States have supplemented the NSPA with their own State counterpart needle safety legislation.

In November, 2010, the International Healthcare Worker Safety Center (the “IHWSC”) at the University of Virginia sponsored the conference “Tenth Anniversary of the Needlestick Safety and Prevention Act: Mapping Progress, Charting a Future Path” in Charlottesville, Va. Members of the IHWSC steering committee drafted the “Consensus Statement and Call to Action” for “Moving the Sharps Safety Agenda Forward in the United States” (the “Consensus Statement”). The Consensus Statement noted that sharps injury rates had leveled off but in some areas had increased since 2002, even while recognizing that medical device manufacturers had helped introduce a broad range of innovative safety-engineered products and noting that the sharps injury rates had initially dropped in the first two years (2001 and 2002) after the NSPA.

For example, IHWSC's Exposure Prevention Information Network Data Reports (“EPINET Data Reports”) indicate that the overall sharps incidents decreased from 39% in 1999 to 22% in 2001 (a 44% decrease), then increased to 29% by 2007 (a 27% increase), then decreased to 20% by 2009 (a 31% decrease).

Narrowing the dataset to only “central-line catheters,” “other non-vascular catheters” and “trocars” from the EPINet Data Reports reveals trends that are more relevant to the present invention. Although comprising a smaller percentage of overall sharps incidents, the narrowed selection indicates an overall variably increasing trend of sharps incidents compared to the overall group (2.6% increasing to 9.5% of the overall group of sharps incidents). Comparing the narrowed selection to the overall group in the EPINet Data Reports indicates a similar initial reduction in 2001, decreasing from 1% in 1999 to 0.47% in 2001 (a 53% decrease). Thereafter, the narrowed group continually increased (as opposed to the overall group), increasing to 1.6% by 2007 (a 236% increase) then increasing again to 1.9% by 2009 (a 21.5% increase).

Moreover, it has been documented that, while there have been promising improvements in adopting safety devices, there remain many unmet needs for improved safety device technologies, although mainly in the clinical (non-acute care) setting. (Hogan Amber. Gaps and successes of safety device market conversion. Materials Management in Health Care. 2005; 14:33-4)

In the absence of any major safety related device advances observed in the marketplace for safety sharps instruments (such as safety trocars), sharps injuries from both handling and using such instruments have been largely reduced only through procedural improvements and education in proper techniques, existing engineering controls and best practices.

Still, the detrimental impact of sharps injuries is most profound regarding potential infection by the hepatitis B virus (“HBV”), hepatitis C virus (“HCV”), human immunodeficiency virus (“HIV”), and other communicable diseases. Lawsuits, clinician or patient injury and disease treatment boost insurance costs for both patients and hospitals. The health and treatment costs of sharps injuries may justify improved safety trocar devices that minimize or eliminate these injuries and reduce costs to hospitals and insurers. The Center for Disease Control (“CDC”) has identified desirable guidelines as well as the need for enhanced safety devices. The CDC offers a summary of guidance at: http://www.cdc.gov/HAI/prevent/prevent_pubs.html, and a sharps safety workbook at https://www.premierinc.com/safety/topics/needlestick/cdc-sharps-injury-prevention.jsp

While attempts have been made to improve safety trocars and similar sharps instruments, the existing devices do not offer a complete solution to the problems of effective and cost-effective devices that impart patient and clinician safety nor to the unmet requirements for clinician ease-of-use and low-cost manufacturability, as demonstrated by a lack of a widespread solutions and limited degrees of market success in the field. There remains an unsatisfied need for sound safety advances in safety sharps instruments such as safety trocar devices.

III. Description of the Related Art

Some attempts have been made to enhance the safety and handling of trocar devices and devices intended to deploy trocars and reduce sharps injuries that may occur during their use.

U.S. Pat. No. 6,613,039 and U.S. Pat. No. 7,938,810 and U.S. Pat. Application No. 2011/0172600 describe a “Safe Trochar with Guide for Placement of Surgical Drains” in which a “method and apparatus for the safe surgical placement of trochars” are described, wherein a guide mechanism holds a trocar and receiving mechanism receives the trocar.

U.S. Pat. No. 7,909,802 describes a “Device for Inserting a Drain and Handle for Such a Device” in which a needle, a drain and a handle are used to insert a drain into a wound, wherein the needle has “a sharp front end effective for perforating a skin, the drain being connected to a rear end of the needle, the handle having a hand-grip and a needle-grip and an exit path for the drain.” In its background descriptions, this patent refers to devices for inserting a drain into a wound: “Device for Inserting a Drain into a Wound” as described in international patent application WO 98/23321; a “Spike for Transcutaneous Lead for Drainage Tube” as described in German patent application DE000004416976A1; “Medical Tissue Drainage Device” as described in European patent application 0 623 355; and “Surgical Needle [and] into Them [a] Piece of Drainage Tube” as described in Dutch patent application 72 16 160.

U.S. Pat. No. 5,607,405 describes a “Surgical Insertion Device and Method” in which a surgical insertion device “includes a trocar with a pierceable sheath covering the forward point, a handle removably affixed to the rear end, and a notch formed on the trocar just behind the point. The notch facilitates gripping of the trocar with a tool during removal of the trocar from the patient's body. Optionally, a kit including the insertion apparatus is further provided with a suitable such tool.”

U.S. Pat. Application No. 2011/0118673 describes a “Needle Safety Cap” as a “safety cap for use with a needle, the cap including an opening in the cap adapted to receive a needle therein, a releasable lock mechanism disposed within the opening, and a permanent lock mechanism disposed within the opening. The safety cap may be provided on a needle, the releasable locking mechanism being actuated to release the needle from the cap. After use, the needle may be reinserted into the safety cap and permanently locked therein by the permanent locking mechanism, thereby providing for safe disposal of the used needle.” The needle is described as a trocar.

U.S. Pat. Application No. 2009/0204140 describes a “Surgical Trocar” that includes a “trocar needle with a sharp tip for puncturing and passing through the skin of a patient . . . . A head formed aft of the tip of the trocar needle has a locking ridge to provide a grip” and additionally provides for a receiver for gripping the trocar needle.

While each of the above mentioned devices may be useful to some degree, they all bear certain disadvantages including complexity of design or requirements, cumbersome usage, and lack of various safety features.

An advantage of the present invention is to implement features that provide for simplicity and ease-of-use via a compact and agile tool, comprised of a relatively few number of component parts. An advantage of the present invention is an easy-to-manipulate dispenser that the clinician may hold with one hand to circumnavigate within the tight spacing among organs within a surgical site, together with the corresponding hand-grip receiver that the clinician holds with the other hand as a backstop and target for the emerging trocar tip.

An advantage described in one embodiment of the receiver of the present invention is the use of a locking mechanism that is positioned within the receiver in an innovative, low-cost manner without the need for expensive or complex manufacturing steps. One embodiment uses a plate aperture and spring approach; a second embodiment uses physically resistive protuberances; a third embodiment uses active gripping rollers. A further advantage of the present invention is that, once the trocar tip is inserted within the receiver, a locking mechanism actively exerts gripping pressure on the trocar shaft when the trocar is pulled in the direction of extraction out of the receiver. In this way, the trocar is permanently locked and safely covered within the receiver.

The present invention advantageously implements an internal coupler that is customizable to adapt and grip presently existing designs of various trocars within the dispenser handle, without requiring further customization of the dispenser handle or the trocar itself. A further advantage of the present invention is an internal channel to conduct the drainage tubing within the dispenser handle; this channel is advantageous to minimize interference between the clinician's hand while holding the handle and the conduction of the drainage tubing.

Further, while inserting the trocar tip into the receiver, a large, removable shield on the receiver protects the clinician's hand as well as provides a backstop and target to manually guide the tip into the center of the receiver. Because the large shield can be removed from the receiver hand-grip locking the trocar, the compact-sized single-use receiver (and locked trocar) is discarded in a small sharps disposal container while the large shield is discarded in any receptacle for contaminated objects. After extricating the distal end of the drainage tubing from the dispenser handle, the dispenser handle is also discarded into a contaminated waste receptacle.

An advantage of the present invention is an easily extendable or retractable sheath that covers the trocar. The extended sheath protects both the clinician and patient from the sharp tip before and during deployment of the trocar. The sheath is locked in the extended position until the clinician positions the sheath within the lumen (i.e., the surgical cavity within the surgical site) and is ready to insert the trocar into the patient's tissue, at which time the clinician manually releases the sheath to permit its retraction.

### BRIEF SUMMARY OF THE INVENTION

A device has been invented to minimize needle-stick and sharps injuries that may result from handling and using sharp instruments during open-site procedures, such as trocars and needles used to introduce wound drains or certain catheters. A preferred embodiment of the invention is a device used to deploy a wound drain using a trocar during open-site surgeries. The invention provides benefits to both patients as well as clinicians by incorporating features that improve patient and clinician safety as well as provide cost-effectiveness.

In the preferred embodiment, the invention comprises a dispenser handle to hold a trocar; a sheath to protect the trocar sharp tip and shaft until inserting the trocar into a patient's skin; and a receiver that captures and traps the trocar tip after the tip emerges from the patient, thereafter safely locking the trocar tip and facilitating disposal of the trocar into a sharps container.

In the preferred embodiment, the dispenser handle incorporates an ergonomic shape to facilitate handling and inserting the trocar through the patient's skin, enabling the clinician to manipulate the instrument within the surgical site and to exert effectively the force needed to push the trocar through the patient's tissue. The dispenser handle incorporates a protective edge-lip that partially covers the clinician's hand when the clinician probes the surgical site, and asserts tactile resistance when the clinician pushes the trocar into and through the skin of the patient.

The sheath covers the trocar during all steps before deploying the trocar within the surgical site, which may include assembly, shipping, and storage. The sheath continually covers the trocar and does not retract to expose the trocar tip until the clinician positions the sheath against the patient's skin and depresses a safety trigger button, simultaneously pushing the trocar into and through the skin. Because the sheath keeps the trocar tip covered, there is no danger of inadvertently puncturing the patient's tissues or organs while the clinician probes the surgical site to find the desired insertion point.

A receiver is used to capture the trocar after it penetrates through the skin and exits the wound site. The receiver is fashioned with a receiver handle grip and a large protective shield both to provide a tactile backstop for the clinician and to protect the clinician's hand.

First, while guiding the dispenser handle within the surgical site, the clinician presses the receiver and shield against the outside of the patient's skin to provide a backstop and target. The clinician finds the backstop and positions the sheath on the inside of the surgical site against the patient's tissues.

Second, the clinician inserts the trocar through the tissues and guides the trocar tip into the receiver. The large shield protects the clinician's hand when the clinician guides the sharp trocar tip through the wound opening and into the receiver. The shape of the removable shield in the preferred embodiment is fashioned as a funnel to actively guide the tip into the center of the receiver's aperture and locking mechanism, where the tip becomes trapped.

Third, since the trocar tip is safely and permanently locked within the receiver, the clinician withdraws the receiver together with the embedded trocar and a segment of the adjoining drainage tubing through the wound opening and out of the patient's skin, without risk of slippage or injury due to the safely covered trocar tip.

Fourth, after extricating the trocar and a segment of drainage tubing out of the surgical site, the clinician cuts the drainage tubing at a point behind the stem of the trocar. The clinician then positions as desired the end of the tubing remaining within the surgical site as a wound drain to collect and discharge bodily fluids.

In addition to safety features, the preferred embodiment incorporates cost-effectiveness by manufacturing with a minimal number of injection-molded plastic components and a minimal number of easily-fabricated metal parts.

### BRIEF DESCRIPTION OF THE DRAWINGS

FIG. 1 depicts an exploded perspective view of the Trocar Dispenser and Safety Grip Receiver System.

FIG. 2 depicts an exploded perspective view of the dispenser portion of the invention.

FIG. 3 depicts dispenser handle side cutaway section view

FIG. 4 depicts dispenser handle, coupler and sheath—side elevation view and frontwards-looking cross section view

FIG. 5 depicts coupler—backwards-looking elevation view, side elevation view, top cutaway view

FIG. 6 depicts dispenser handle, coupler and sheath extended—top elevation view, side cutaway view, top cutaway view, enlargement

FIG. 7 depicts dispenser handle, coupler and sheath retracted—top cutaway view, enlargement

FIG. 8 depicts coupler, opt 1—backwards-looking elevation view, side elevation view, top cutaway view

FIG. 9 depicts dispenser handle, coupler and sheath extended, opt 1—top cutaway view, enlargement

FIG. 10 depicts dispenser handle, coupler and sheath retracted, opt 1—top cutaway view, enlargement

FIG. 11 depicts coupler, opt 2—backwards-looking elevation view, side elevation view, top cutaway view

FIG. 12 depicts dispenser handle, coupler and sheath extended, opt 2—top cutaway view, enlargement

FIG. 13 depicts dispenser handle, coupler and sheath retracted, opt 2—top cutaway view, enlargement

FIG. 14 depicts sheath—backwards-looking elevation view, side elevation view, top elevation view, side cutaway view

FIG. 15 depicts an exploded perspective view of the receiver portion of the invention

FIG. 16 depicts a receiver handle grip—backwards-looking elevation view, side cutaway view

FIG. 17 depicts a receiver coupler—bottom elevation view

FIG. 18 depicts a receiver coupler—backwards-looking elevation view

FIG. 19 depicts a receiver coupler—top elevation view

FIG. 20 depicts a receiver coupler—bottom cutaway view

FIG. 21 depicts a receiver coupler—side cutaway view

FIG. 22 depicts a receiver—side cutaway view

FIG. 23 depicts a receiver, trocar inserted—side cutaway view

FIG. 24 depicts a locking plate—backwards-looking elevation view, side cross section view

FIG. 25 depicts a receiver, opt 1—exploded perspective view

FIG. 26 depicts a receiver, opt 1—side cutaway view

FIG. 27 depicts a receiver, trocar inserted, opt 1—side cutaway view

FIG. 28 depicts a locking plate, opt 1—backwards-looking elevation view, side cross section view

FIG. 29 depicts a receiver, opt 2—exploded perspective view

FIG. 30 depicts a receiver coupler, opt 2—backwards-looking elevation view

FIG. 31 depicts a receiver coupler, opt 2—top elevation view

FIG. 32 depicts a receiver coupler, opt 2—side elevation view

FIG. 33 depicts a receiver, opt 2—side cutaway view

FIG. 34 depicts a receiver, trocar inserted, opt 2—side cutaway view

FIG. 35 depicts a locking plate, opt 2—backwards-looking elevation view

FIG. 36 depicts a locking plate, opt 2—top elevation view

FIG. 37 depicts a locking plate, opt 2—side elevation view

FIG. 38 depicts a locking roller—backwards-looking elevation view, top elevation view

FIG. 39 depicts a shield backwards-looking elevation view, side cutaway view

FIG. 40 depicts a dispenser and receiver system—second exploded perspective view

1—Trocar
2—Trocar tip
3—Trocar shaft
4—Trocar distal end
5—Drainage tubing
6—Dispenser
7—Dispenser handle
8—Dispenser handle lip
9—Dispenser handle aperture
10—Channel for sheath guide rail
11—Channel for coupler guide rail
12—Opening for trigger release button
13—Trigger guard
14—Dispenser handle inner lip back-stop
15—Dispenser handle distal aperture
16—Guide channel for drainage tubing
17—Dispenser handle thumb grip
18—Coupler
19—Coupler guide rail
20—Coupler proximal aperture
21—Coupler distal aperture
22—Coupler feet
23—Dispenser handle interference #1 to coupler feet
24—Dispenser handle interference #2 to coupler feet
25—Coupler—opt 1
26—Coupler guide rail—opt 1
27—Coupler proximal aperture—opt 1
28—Coupler distal aperture—opt 1
29—Coupler feet—opt 1
30—Coupler feet protuberance—opt 1
31—Dispenser handle interference #1 to coupler feet—opt 1
32—Dispenser handle interference #2 to coupler feet—opt 1
33—Dispenser handle interference #3 to coupler feet—opt 1
34—Coupler—opt 2
35—Coupler guide rail—opt 2
36—Coupler proximal aperture—opt 2
37—Coupler feet—opt 2
38—Trocar shaft—option
39—Dispenser handle interference #1 to coupler feet—opt 2
40—Dispenser handle interference #2 to coupler feet—opt 2
41—Sheath
42—Sheath guide rail
43—Slot in sheath
44—Sheath aperture
45—Sheath lip edge
46—Sheath proximal end trocar tip support
47—Sheath trigger release button
48—Sheath stop against inner wall of receiver handle lip
49—Receiver
50—Receiver coupler
51—Receiver handle
52—Locking mechanism
53—Shield
54—Receiver handle grip
55—Receiver handle grip aperture
56—Receiver handle grip outer aperture
57—Receiver handle grip joining-surface circumference
58—Receiver handle grip joining-surface protuberances
59—Receiver handle grip distal circumferential edge
60—Flared base
61—Receiver coupler
62—Receiver coupler outer aperture
63—Receiver coupler inner aperture
64—Receiver coupler axial channel
65—Receiver coupler slot 1 for handle grip
66—Receiver coupler slot 2 for handle grip
67—Receiver coupler top slot
68—Receiver coupler bottom slot
69—Receiver coupler spring channel
70—Locking plate top tab
71—Locking plate bottom tab
72—Locking plate
73—Locking plate angle
74—Locking plate aperture
75—Spring
76—Receiver coupler—opt 1
77—Receiver coupler outer surface—opt 1
78—Receiver coupler outer aperture—opt 1
79—Flared base
80—Receiver coupler inner aperture—opt 1
81—Receiver coupler axial channel—opt 1
82—Receiver coupler slot 1 for handle grip—opt 1
83—Receiver coupler slot 2 for handle grip—opt 1
84—Receiver coupler top slot—opt 1
85—Receiver coupler bottom slot—opt 1
86—Locking mechanism—opt 1
87—Locking plate top tab—opt 1
88—Locking plate bottom tab—opt 1
89—Locking plate—opt 1
90—Locking plate angle—opt 1
91—Locking plate aperture—opt 1
92—Locking plate aperture protuberances
93—Receiver coupler—opt 2
94—Receiver coupler outer surface—opt 2
95—Receiver coupler outer aperture—opt 2
96—Flared base
97—Receiver coupler inner aperture—opt 2
98—Receiver coupler axial channel—opt 2
99—Receiver coupler slot 1 for handle grip—opt 2
100—Receiver coupler slot 2 for handle grip—opt 2
101—Receiver coupler top slot—opt 2
102—Receiver coupler bottom slot—opt 2
103—Locking mechanism—opt 2
104—Plate top bracket leads—opt 2
105—Plate bottom bracket leads—opt 2
106—Plate—opt 2
107—Plate aperture—opt 2
108—Locking rollers
109—Shield proximal surface
110—Shield proximal aperture
111—Shield distal aperture
112—Shield lip

### DETAILED DESCRIPTION OF THE INVENTION

In the description that follows, any reference to either orientation or direction is intended primarily and solely for the purpose of illustration and is not intended in any way as a limitation of the scope of the present invention or its claims. Also, the particular embodiments described herein although being noted as preferred are not to be considered as limiting of the present invention. Furthermore, like-parts or like-elements in the various drawings hereto are identified by like-numerals.

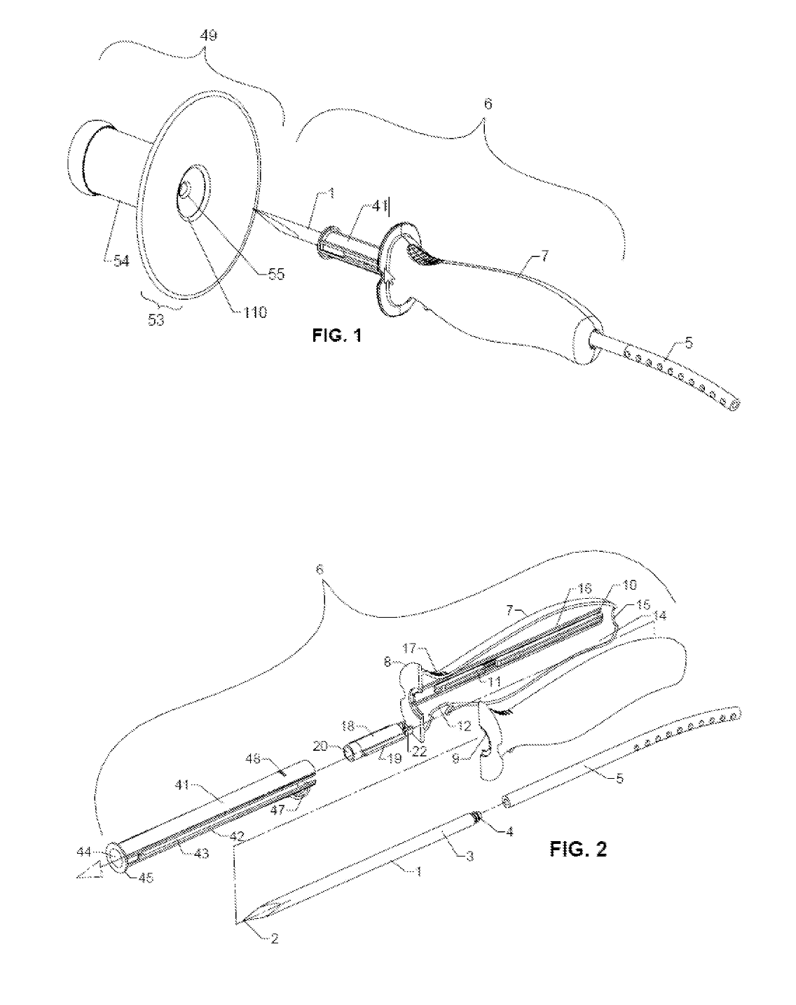

FIG. 1 illustrates the device according to the present invention by an exploded view showing the safety dispenser 6 (showing a dispenser handle 7 and sheath 41) and including the presence of a trocar 1 and drainage tubing 5. This figure also illustrates the safety grip receiver 49 (showing a shield 53 and the shield proximal aperture 110, and a receiver handle grip 54 and the receiver handle grip inner aperture 55). The components of the invention are explained in the following figures and paragraphs.

FIG. 2 illustrates an exploded perspective view of the preferred embodiment of the dispenser 6 according to the present invention, illustrating the various component parts and structure as well as their assembly. Generally the dispenser 6 comprises a rigid, hollow handle 7 with an axial channel 16, a proximal handle aperture 9 and a distal handle aperture 15. The subsequent figures explain the detailed features of the components and structure in greater detail.

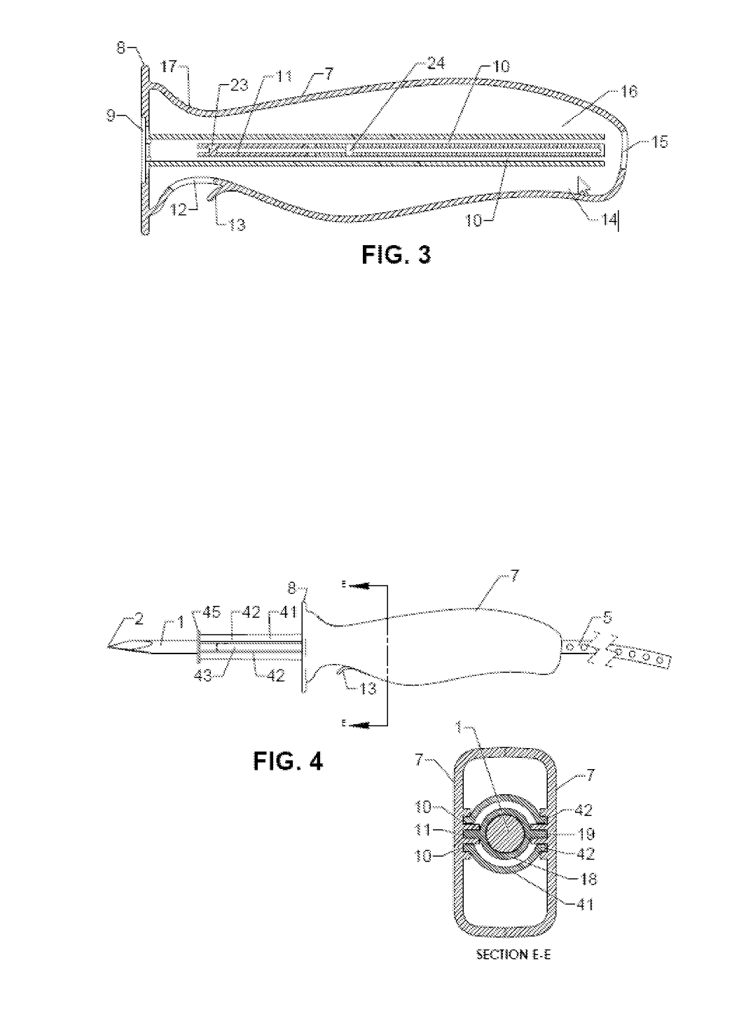

FIG. 3 illustrates a side cutaway view showing half of the dispenser handle 7 (a “handle”), whose features are mirrored identically with the opposite half of the dispenser handle. On the inside wall of the dispenser handle 7 are formed various guide channels to facilitate the internal alignment of the various components. These guide channels include: an internal dispenser axial channel 16 that is comprised of the hollow interior of the dispenser handle 7, which conducts drainage tubing 5 in a generally straight path from the dispenser handle distal aperture 15 to the dispenser handle proximal aperture 9; a pair of internal guide channels 10 that is approximately 1.5 mm in depth to conduct a pair of sheath guide-rails 42 (discussed below); and an internal guide channel 11 that is approximately 1.3 mm in depth to conduct a coupler guide-rail 19 (discussed below). The depth of the guide channel 11 for the coupler guide rail 19 gradually decreases at the termination point at the distal end of the guide channel 11, terminating in an interference feature 24. At the proximal end of the guide channel 11, the channel terminates in a second interference feature 23.

The inner diameters of the hollow dispenser handle axial channel 16 and proximal dispenser handle aperture 9 are sufficient to accommodate a coupler 18 (discussed below) and a sheath 41 (discussed below) that encompasses the coupler 18, hence the proximal dispenser handle aperture 9 is selected from a range of between 0.3 mm to 25 mm in diameter, preferentially 15 mm in diameter. The length of the dispenser handle 7 is sufficient to encompass the sheath 41 that may be fully retracted within the dispenser handle 7, and the trocar 1 within it, hence is selected from a range of 40 mm to 300 mm and is preferentially 130 mm. The inner diameters of the hollow dispenser handle axial channel 16 and distal dispenser handle aperture 15 are sufficient to accommodate drainage tubing 5.

The outside height of the dispenser handle 7 in the vertical direction shown in FIG. 3 is formed in a generally bulbous shape, curving with a backwards-reclining “s” curve. The resultant shape permits a clinician to hold the dispenser handle 7 in a relaxed, neutral position in the wrist and fingers. The proximal end of the dispenser handle 7 that is gripped by the thumb and forefinger measures approximately 15 mm to 35 mm, preferentially 20 mm, which is relatively narrower in diameter compared to the midsection of the dispenser handle 7. The midsection is the thickest portion of the dispenser handle 7 and comprises an outer diameter that may be comfortably gripped in the palm of a clinician's hand, approximately 25 mm to 50 mm, preferentially 35 mm. Towards the distal end, the dispenser handle 7 tapers inwards to a narrower diameter relative to the midsection and measures approximately 20 mm to 30 mm, preferentially 25 mm.

The tapering at the distal end of the dispenser handle 7 and, along the lower edge, the downwards curve in a reclining “s” curved shape provides a compressed and slanted inner surface lip area 14 of the dispenser handle 7, such that the inner surface 14 of the dispenser handle 7 is slanted at an acute angle, measured from direction parallel to the axial channel direction and towards the distal end of the dispenser handle 7, which provides a designed-in lip 14 to backstop a sheath trigger release button 47 (discussed below).

The proximal end of the dispenser handle 7 comprises a flared shape and dispenser handle lip 8 behind which the clinician's hand would enclose the dispenser handle 7. The flared shape lends support to the dispenser handle lip 8 and is shaped at an inclined slope (measured from the direction parallel to the axial channel in the proximal direction and towards the proximal end of the dispenser handle 7) of approximately 45 degrees, so that the opening of the clinician's hand between his thumb and forefinger may comfortably grip the dispenser handle 7 behind the dispenser handle lip 8. The outside diameter of the dispenser handle lip 8 extends in a preferentially elliptical shape in the direction transverse to the axial channel beyond the edges of the clinician's hand so that the dispenser handle lip 8 provides support when the clinician pushes against it. It is contemplated an alternative shape also comprises a circle, rectangle, or other shapes. The width and height of the dispenser handle lip 8 ranges from 10 mm to 50 mm, preferentially a width of 32 mm by a height of 45 mm, affording an overlap section (measured from the edge of the dispenser handle proximal aperture 9 to the edge of the dispenser handle lip 8) of preferentially 32 mm in the height direction and 45 mm in the width direction. A section along the top 17 of the dispenser handle 7, at the proximal end behind and adjacent to the dispenser handle lip 8, comprises a texturized surface for a thumb grip 17 upon which the clinician can optionally position and push with his thumb.

In the preferred embodiment, at the bottom of the dispenser handle 7 at the proximal end immediately behind the dispenser handle lip 8 is an opening 12 that accommodates the sheath trigger release button 47. At the bottom of the dispenser handle 7, behind (in the distal direction) the dispenser handle lip 8 and trigger opening 12 is a stub that forms a trigger guard 13. The clinician rests his finger on this trigger guard 13 while guiding the dispenser handle 7 and trocar 1 in Disarmed Mode (defined below) within the surgical site but before exposing the trocar tip 2 in Armed Mode (defined below). Preferentially, the surface of the trigger guard 13 and the area adjacent to it in the distal direction are formed with a texturized surface similar to the thumb grip 17. It is contemplated an alternative embodiment for the position of the trigger guard 13 is behind the dispenser handle lip 8 (in the distal direction), between the dispenser handle lip 8 and the trigger opening 12. It is also contemplated an alternative embodiment for the dispenser handle 7 is to fabricate it without a trigger opening 12 or a trigger guard 13.

For ease of manufacturing, the dispenser handle 7 is molded into two halves, into which the inner components are aligned and placed (without requiring any fixation), and then the second half of the dispenser handle 7 is joined and fixated with an appropriate adhesive means known in the art.

FIG. 4 illustrates a side view and a frontwards-looking cross section E-E view. The side view in FIG. 4 illustrates a trocar 1, which is positioned within a sheath 41 (discussed below), which is positioned within the dispenser handle 7. FIG. 4 also shows features that will be discussed below, including the sheath lip 45, sheath guide rails 42, and sheath slot 43.

The frontwards-looking cross section E-E view in FIG. 4 illustrates the trocar 1, which is positioned within a coupler 18 (discussed below), which is positioned within the sheath 41, which is positioned within the dispenser handle 7. Further, the frontwards-looking section view illustrates the sheath guide channels 10 accommodating the sheath guide rails 42, the sheath guide channel 11 accommodating the coupler guide rail 19, and the sheath's interleaved slot 43 accommodating both the coupler guide channel 11 and coupler guide rail 19.

The frontwards-looking cross section E-E view in FIG. 4 further illustrates the horizontal profile of the dispenser handle 7, which consists of a constant width measuring approximately 15 mm to 30 mm, preferentially 20 mm.

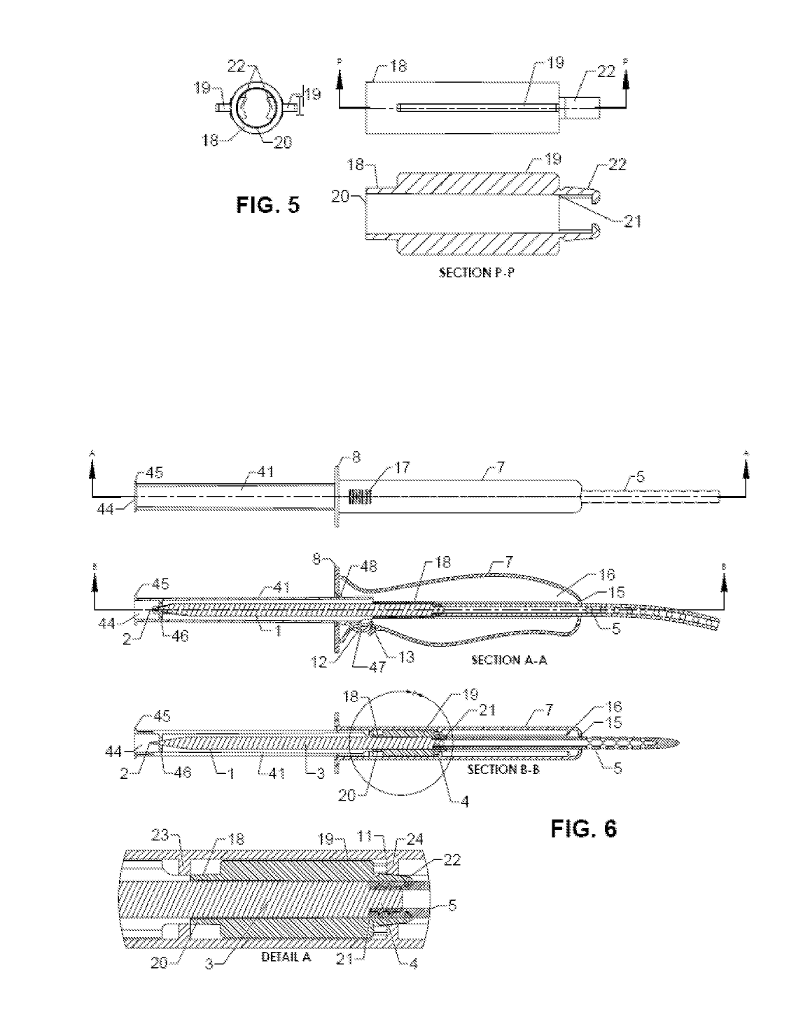

FIG. 5 illustrates a backwards-facing elevation view, side elevation view, and top cutaway section P-P view of the preferred embodiment of the coupler 18. The coupler 18 is generally a rigid, cylindrical encasement with a hollow axial channel and an aperture at the proximal 20 and distal 21 ends through which a trocar 1 is passed. Since the invention is designed to accommodate a trocar whose diameter may range from 0.5 mm to 15 mm, the diameter of the coupler proximal aperture 20, coupler distal aperture 21, and the axial channel of the coupler 18 is selected from a range of 0.5 mm to 15 mm and preferentially from 3 mm-7 mm (plus any minimal manufacturing tolerance clearances). By fabricating a coupler aperture 20, 21 and axial channel with a particular diameter, therefore, the coupler 18 acts as an adapter that is selected to fit a particular trocar 1 size.

This figure also shows extending along the full-length in a direction parallel to the axial channel of the coupler 18 on two diametrically opposed outside surfaces are two coupler guide rails 19 that are each approximately 1.3 mm in height. These coupler guide rails 19 are formed to and fit into the corresponding channels 11 formed on the inner surface of the dispenser handle 7. At the distal end of the coupler 18 extending beyond the coupler distal aperture 21 in the distal direction, the coupler guide rails 19 extend into two hinged coupler feet 22 that grip the trocar distal end 4. At the distal end of each of the coupler feet 22 is formed an inwardly facing pronged protuberance that presses against the ribbed or other type of connector (an “adjoining geometry”) at the trocar distal end 4, which connects drainage tubing 5 to the trocar 1.

FIG. 6 illustrates various views of a dispenser handle 7 encompassing an extended sheath 41, a trocar 1 protected within the sheath 41 in the Disarmed Mode (defined below), and drainage tubing 5. A top elevation view shows the dispenser handle 7, the extended sheath 41, and drainage tubing 5; a side cutaway section A-A view and top cutaway section B-B view each shows the dispenser handle 7, the extended sheath 41, the protected trocar 1, a coupler 18, and the trailing drainage tubing 5; an enlargement detail A of the top cutaway section B-B view highlights the detail of the trocar shaft 3 and its distal end 4, the coupler 18, and the trailing drainage tubing 5.

The side cutaway section A-A view illustrates the preferred embodiment for the position of the trigger guard 13, adjacent (in the distal direction) to the opening 12 (a “handle catch release mechanism”) for the trigger release button 47 (a “sheath catch release mechanism”), at the proximal end of the dispenser handle 7.

The top cutaway section B-B view and enlargement detail A illustrate the preferred embodiment for the gripping mechanism, showing the coupler feet 22 gripping the distal end 4 of the trocar shaft 3. The handle guide channel 11 terminates in the distal direction at interference stub 24 (a “handle catch-mechanism”), acting as a backstop that stops each of the coupler guide rails 19 (a “coupler catch mechanism”) and prevents the coupler 18 from moving further backwards (in the distal direction) into the dispenser handle 7. The decreasing depth of the dispenser handle guide channel 11 at interference stub 24 also pushes the coupler feet 22 inwards, inducing inwards pressure upon the ribbed end at the trocar distal end 4 where the trocar 1 connects to drainage tubing 5, and grips the trocar firmly. Since the coupler 18 is stopped from moving further backwards (in the distal direction) into the dispenser handle 7, the gripped trocar 1 is also stopped from moving further backwards.

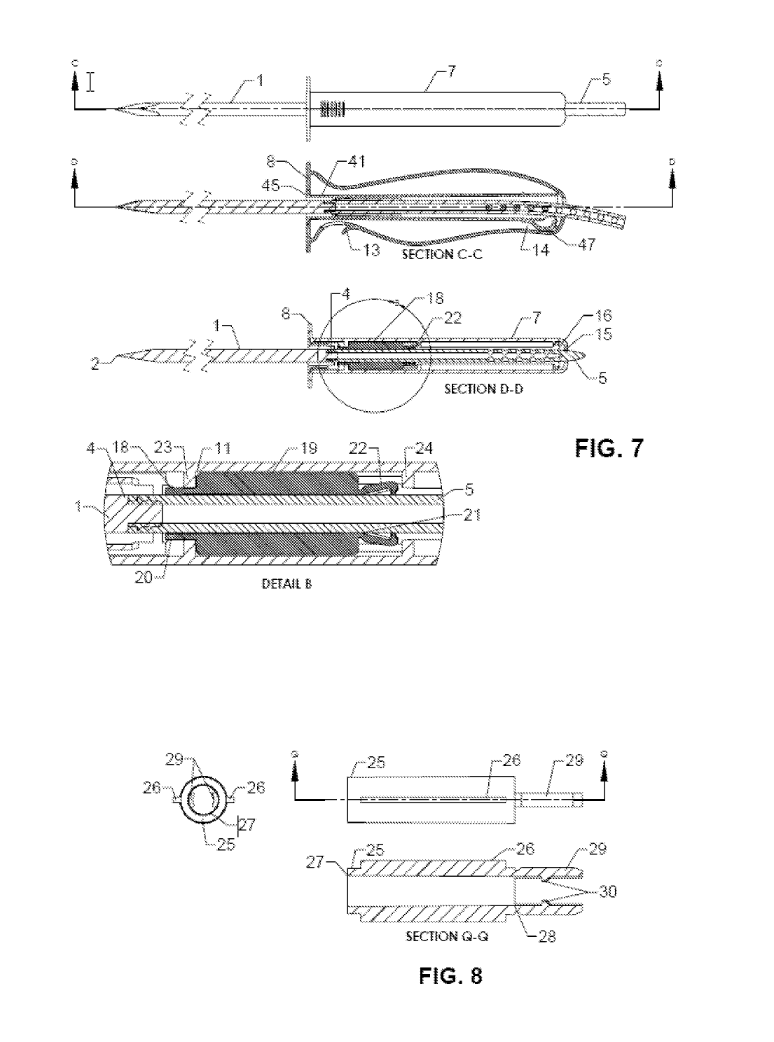

FIG. 7 illustrates various views of a dispenser handle 7 encompassing a fully-retracted sheath 41, a trocar 1 extended out of the sheath 41 and dispenser handle 7 in the Armed Mode (defined below), and drainage tubing 5. A top elevation view shows the dispenser handle 7, the trocar 1, and drainage tubing 5; a side cutaway section C-C view and top cutaway section D-D view each shows the dispenser handle 7, the retracted sheath 41, the exposed trocar 1, a coupler 18, and the trailing drainage tubing 5; an enlargement detail B of the top cutaway section D-D view highlights the detail of the trocar 1 and its distal end 4, the coupler 18, and the trailing drainage tubing 5. The above features notwithstanding, it is also contemplated that an alternative trocar backstop could be formed at the distal end within the dispenser handle 7 without requiring the implementation of a coupler 18.

The side cutaway section C-C view illustrates the sheath lip edge 45 (a “sheath catch mechanism”) of the fully retracted sheath 41 pressed against the dispenser handle lip 8 (a “handle catch mechanism”), thereby preventing the sheath 41 from retracting further into the dispenser handle 7. The side cutaway section C-C view also illustrates the sheath trigger release button 47 locked against the dispenser handle inner backstop 14 by friction, which prevents the sheath 41 from extending, absent a clinician pulling the sheath with force sufficient to overcome the friction.

The top cutaway section D-D view and enlargement detail B illustrate the preferred embodiment for a coupler guide rail 19 stopped at the interference stub 23 (a “handle catch-mechanism”) at the proximal end of the dispenser handle coupler guide channel 11, which catches on the proximal edges of the coupler guide rails 19 and therefore prevents the coupler 18 from exiting the dispenser handle 7 in the proximal direction. Because each coupler foot 22 has cleared the interference stub 24, the coupler feet 22 are released from squeezing the trocar distal end 4 and the coupler feet 22 no longer grip the trocar 1. Therefore, while the coupler 18 is stopped within the dispenser handle 7, the trocar 1 and drainage tubing 5 can be pulled through the coupler proximal aperture 20 and distal aperture 21 and completely out of the dispenser handle 7.

FIG. 8 illustrates a backwards-facing elevation view, side elevation view, and top cutaway section Q-Q view of an alternative embodiment of a coupler 25, which is implemented similarly to the preferred embodiment except for the coupler feet 29. The coupler 25 is generally a rigid, cylindrical encasement with a hollow axial channel and an aperture at the proximal 27 and distal 28 ends through which a trocar 1 is passed. Since the invention is designed to accommodate a trocar whose diameter may range from 0.5 mm to 15 mm, the diameter of the coupler proximal aperture 27, coupler distal aperture 28, and the axial channel of the coupler 25 is selected from a range of 0.5 mm to 15 mm (plus any minimal manufacturing tolerance clearances). By customizing the coupler apertures 27, 28 and axial channel with a particular diameter, the coupler 25 acts as an adapter that is selected to fit a particular trocar size.

Extending along the full-length in a direction parallel to the axial channel of the coupler 25 on two diametrically opposed outside surfaces are two coupler guide rails 26 that are each approximately 2.5 mm in height. Each coupler guide rail 26 is formed to fit into the corresponding channel 11 formed on the inner surface of the dispenser handle 7. At the distal end of the coupler 25 extending beyond the distal coupler aperture 28, the coupler guide rails extend into two hinged coupler feet 29 in the distal direction, which grip the trocar distal end 4. Each of the feet 29 contains an inwardly facing pronged protuberance 30 on the inner surface near the midsection of the foot 29, which presses against an orifice or notch formed at the trocar distal end 4.

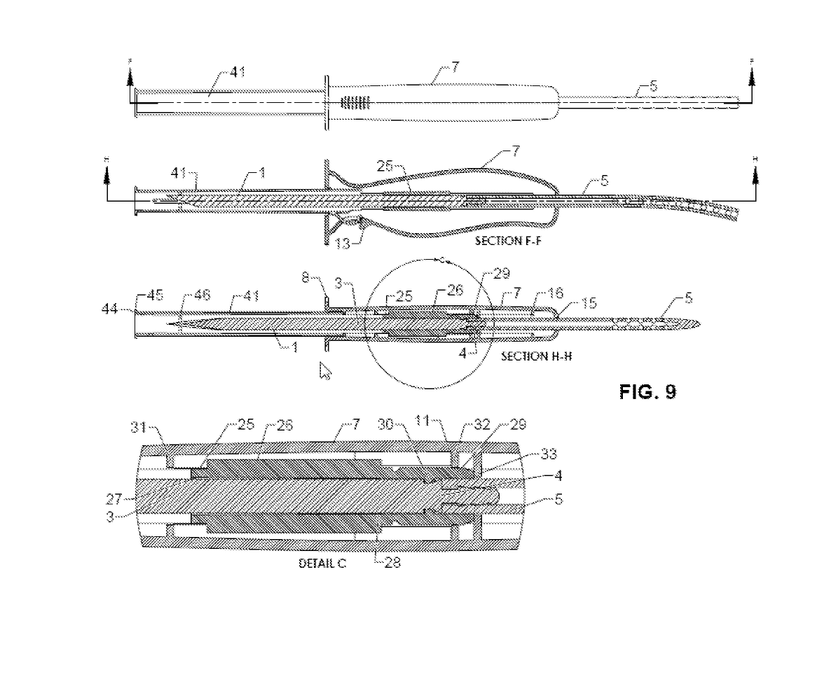

FIG. 9 illustrates various views of a dispenser handle 7 encompassing an extended sheath 41, a trocar 1 protected within the sheath 41 in the Disarmed Mode (defined below), and drainage tubing 5. A top elevation view shows the dispenser handle 7, the extended sheath 41, and drainage tubing 5; a side cutaway section F-F view and top cutaway section H-H view show the dispenser handle 7, the extended sheath 41, the protected trocar 1, the alternative embodiment of the coupler 25, and the trailing drainage tubing 5; an enlargement detail C of the top cutaway section H-H view highlights the detail of the trocar shaft 3 and its distal end 4, the coupler 25, and the trailing drainage tubing 5.

The top cutaway section H-H view and enlargement detail C illustrate the alternative embodiment for the gripping mechanism between the coupler feet 29 and the trocar distal end 4. The dispenser handle coupler guide channel 11 terminates at interference stub 33, acting as a backstop that stops the coupler feet 29 and prevents the coupler 25 and trocar 1 from moving further backwards (in the distal direction) into the dispenser handle 7. The decreasing depth of the dispenser handle coupler guide channel 11 near the channel's distal termination point (at interference stub 32) also pushes the coupler feet 29 inwards, inducing inwards-facing pressure by coupler feet 29 and the coupler feet protuberances 30 upon an adjoining orifice or notch located at the trocar's distal end 4, and grips the trocar 1 firmly. Since the coupler 25 is stopped from moving further backwards (in the distal direction) into the dispenser handle 7, the gripped trocar 1 is also stopped from moving further backwards.

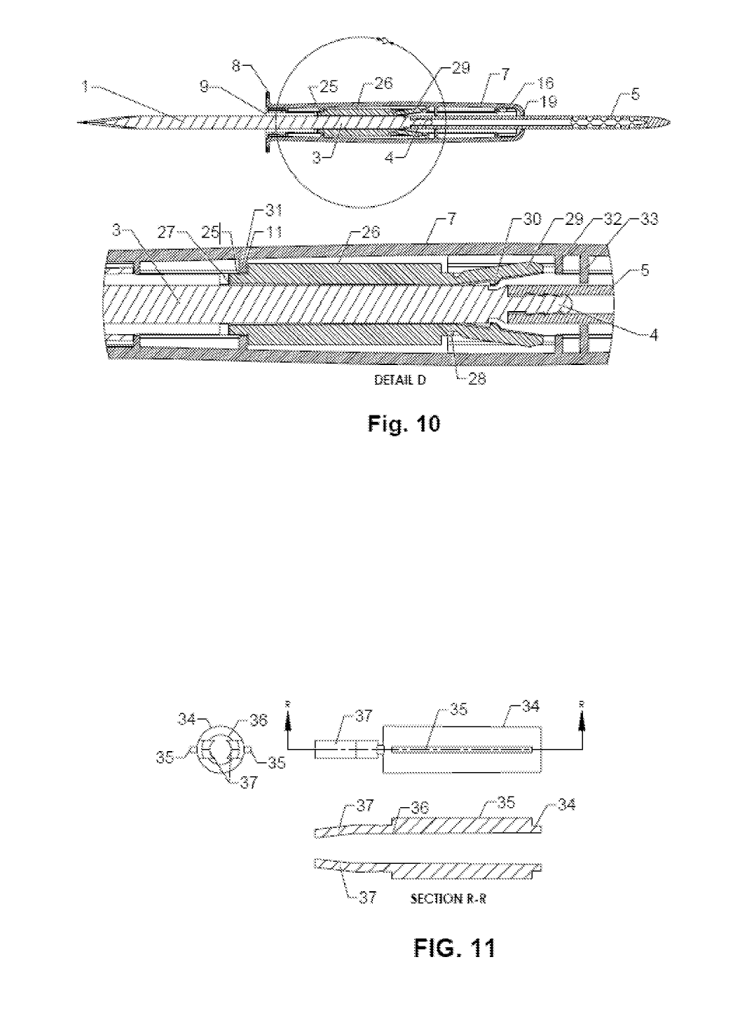

FIG. 10 illustrates various views of a dispenser handle 7 encompassing a fully-retracted sheath 41, a trocar 1 extended out of the sheath 41 and dispenser handle 7 in the Armed Mode (defined below), and drainage tubing 5. A top cutaway view show the dispenser handle 7, the retracted sheath 41, the exposed trocar 1, a coupler 25, and the trailing drainage tubing 5; an enlargement detail D of the top cutaway view highlights the detail of the trocar shaft 3 and its distal end 4, the coupler 25, and the trailing drainage tubing 5. The top cutaway view and enlargement detail D view each illustrates the alternative embodiment for the coupler guide rail 26 stopped at the interference stub 31 at the proximal end of the dispenser handle inner channel 11. The interference stub 31 at the proximal end of the dispenser handle channel 11 catches on the proximal edges of the coupler guide rails 26 and prevents the coupler 25 from exiting the dispenser handle 7 in the proximal direction. Because the coupler feet 29 have cleared the interference stub 32, the coupler feet 29 are released from squeezing the trocar distal end 4, and the protuberances 30 on the bottom of the coupler feet 29 no longer grip the trocar's distal end 4. Therefore, while the coupler 25 is stopped within the dispenser handle 7, the trocar 1 and drainage tubing 5 can be pulled through the coupler proximal aperture 27 and distal aperture 28 and completely out of the dispenser handle 7.

FIG. 11 illustrates a backwards-facing elevation view, side elevation view, and top cutaway section R-R view of a second alternative embodiment of a coupler 34, which is implemented similarly to the preferred embodiment except for the coupler feet 37. The coupler 34 is generally a rigid, cylindrical encasement with a hollow axial channel and an aperture 36 at the proximal and distal ends through which a trocar 1 is passed. Since the invention is designed to accommodate a trocar whose diameter may range from 0.5 mm to 15 mm, the diameter of the coupler proximal and distal apertures 36 and the axial channel of the coupler 34 is selected from a range of 0.5 mm to 15 mm (plus any minimal manufacturing tolerance clearances). By customizing the coupler aperture 36 and axial channel with a particular diameter, the coupler 34 acts as an adapter that is selected to fit a particular trocar size.

Extending along the full-length in a direction parallel to the axial channel of the coupler 34 on two diametrically opposed outside surfaces are two coupler guide rails 35 that are each approximately 2.5 mm in height. Each coupler guide rail 35 is formed to fit into the corresponding channel 11 formed on the inner surface of the dispenser handle 7. At the proximal end of the coupler 34 extending beyond the proximal coupler aperture 36 in the proximal direction, the coupler guide rails 35 extend into two hinged coupler feet 37, which grip a notch or ridge in a trocar shaft 38 (discussed below).

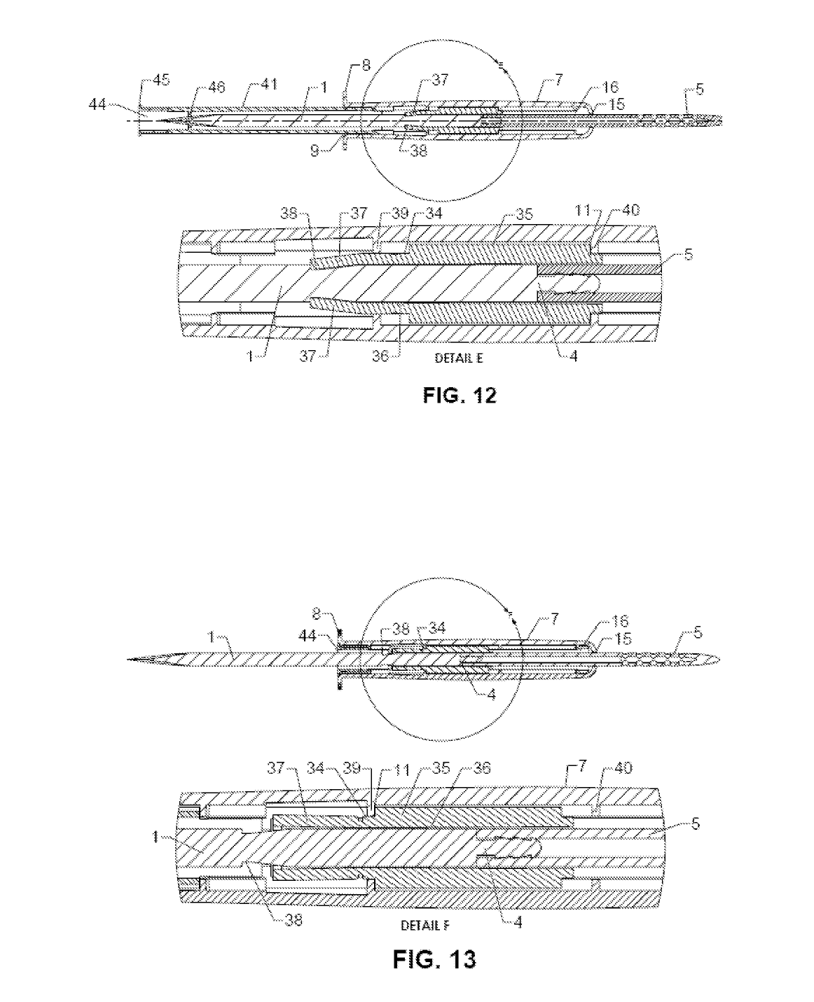

FIG. 12 illustrates a top cutaway view showing the dispenser handle 7 encompassing an extended sheath 41, a trocar 1 protected within the sheath 41 in the Disarmed Mode (defined below), the second alternative embodiment of the coupler 34, and trailing drainage tubing 5. An enlargement detail E of the top cutaway view highlights the detail of the trocar shaft 38, the coupler 34, and the trailing drainage tubing 5.

The top cutaway view and enlargement detail E illustrate the second alternative embodiment for the gripping mechanism between the coupler feet 37 and an orifice or notch formed in the trocar shaft 38. The dispenser handle coupler guide channel 11 terminates in the distal direction at interference stub 40 (a “handle catch-mechanism”), acting as a backstop that stops each of the coupler guide rails 35 (a “coupler catch-mechanism”) and prevents the coupler 34 from moving further backwards (in the distal direction) into the dispenser handle 7. The decreasing depth of the dispenser handle coupler guide channel 11 at the channel's proximal termination point (at interference stub 39) also pushes the coupler feet 37 inwards, inducing inwards-facing pressure upon an adjoining notch or ridge located on the trocar's shaft 38, and grips the trocar firmly. Since the coupler 34 is stopped from moving further backwards (in the distal direction) into the dispenser handle 7, the gripped trocar 1 is also stopped from moving further backwards.

FIG. 13 illustrates a top cutaway view of the dispenser handle 7, a fully-retracted sheath 41, a trocar 1 extended out of the sheath 41 and dispenser handle 7 in the Armed Mode (defined below), a coupler 34, and trailing drainage tubing 5. An enlargement detail F of the top cutaway view highlights the detail of the trocar shaft 38, the coupler 34, and the trailing drainage tubing 5. The top cutaway view and enlargement detail F illustrate the second alternative embodiment for the coupler guide rail 35 stopped at the interference stub 39 at the proximal end of the dispenser handle coupler guide channel 11. The interference stub 39 (a “handle catch-mechanism”) at the proximal end of the dispenser handle coupler guide channel 11 catches on the proximal edge of the coupler guide rail 35 and prevents the coupler 34 from exiting the dispenser handle 7 in the proximal direction. Because the coupler feet 37 have cleared the interference stub 39, the coupler feet 37 are released from squeezing the trocar shaft 38 and the coupler feet 37 no longer grip the trocar shaft 38. Therefore, while the coupler 34 is stopped within the dispenser handle 7, the trocar 1 and drainage tubing 5 can be pulled through the coupler proximal aperture 36 and completely out of the dispenser handle 7.

The above features notwithstanding, it is comtemplated an alternative embodiment for the dispenser 6 is to fabricate it without a coupler 18.

A third alternative embodiment for a structure within the dispenser handle 7 to stop and prevent a gripped trocar from moving further backwards than desired within the dispenser handle 7 comprises a dispenser handle guide channel 16 for the drainage tubing 5 that follows an elbow-bend (a “divergent direction pathway”) within the dispenser handle 7, rather than a straight lengthwise channel within the dispenser handle 7. The angle of the elbow-bend ranges up to 90 degrees, measured from the direction of the axial channel of the dispenser handle 7 in the distal direction, of degree sufficient to block a coupler 34 (or the distal end of a trocar 1 if no coupler 18 is present in the dispenser 6 according to an alternative embodiment) from moving backwards beyond the elbow-bend (a “divergent direction pathway”) within the dispenser handle 7. An alternative exit pathway for the dispenser handle distal aperture 15 other than the rear of the dispenser handle is also contemplated, which would facilitate the elbow-bend of the dispenser handle guide channel 16.

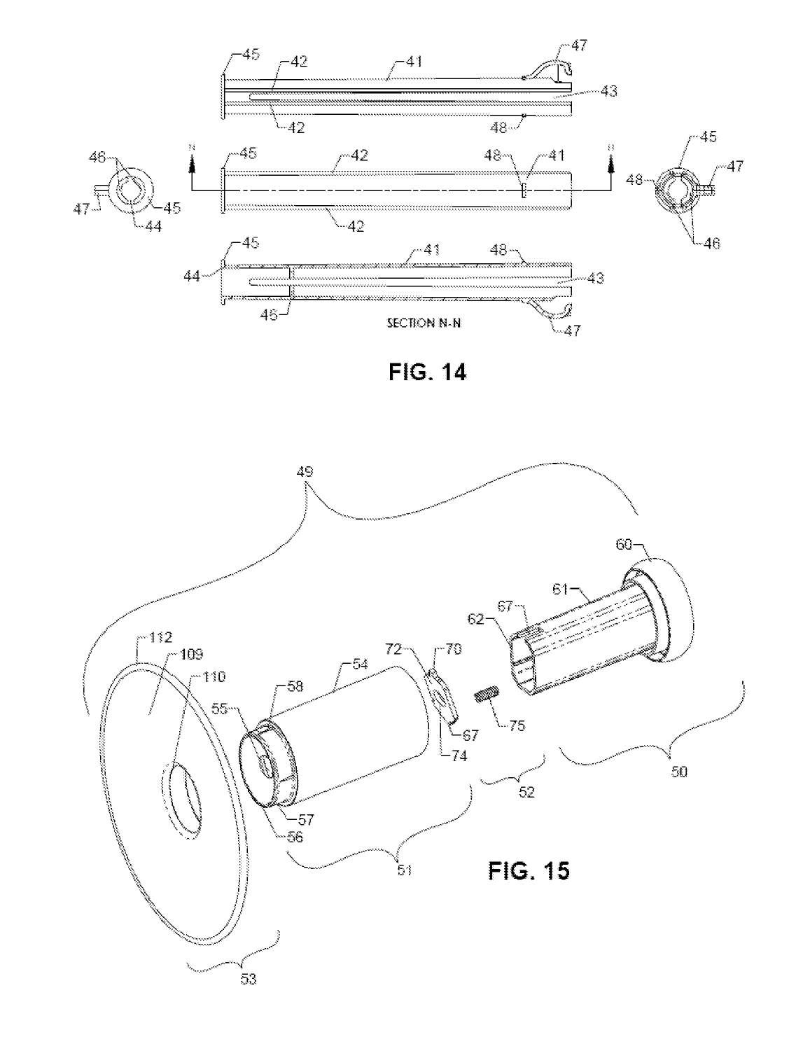

FIG. 14 illustrates a backwards-looking elevation view, upside-down side elevation view, top elevation view, and side cutaway section N-N view of the preferred embodiment of the sheath 41. Generally, the sheath 41 is a rigid, cylindrical encasement with a hollow axial channel (a “sheath channel”) defined by an aperture 44 at the proximal and distal ends and an axial channel through which a trocar 1 is passed. The diameter of the distal and proximal sheath aperture 44 and sheath channel is constant and sized to accommodate a range of trocar diameters plus a clearance gap, hence is selected from a range of between 0.5 mm to 25 mm in diameter and preferentially 10 mm in diameter. The length of the sheath 41 is sufficient to accommodate the length of the trocar 1. Since the invention is designed to accommodate a trocar 1 whose length may range 40 mm to 300 mm, preferentially 150 mm, the length of the sheath 41 is selected from a range of 40 mm to 300 mm and is preferentially 125 mm.

The backwards-looking elevation view illustrates a surface edge lip 45 is formed at the proximal end of the sheath 41, in a direction transverse to the sheath aperture 44 with a width defined as the integral from the edge of the sheath aperture 44 to the outside circumference of the edge lip 45, ranging from 1 mm to 30 mm and preferentially 16 mm.

The upside-down side elevation view shows one of two pairs of rail-guides 42 extending along the full-length of the sheath 41 in a direction parallel to the axial channel on two diametrically opposed outside surfaces of the sheath 41, each preferentially 1.5 mm in height. Each pair of rail-guides 42 fits into the corresponding channels 10 formed on the inner surface of the dispenser handle 7. Situated between the pair of rail-guides 42 is a gap 43, preferentially 3.5 mm in width. The gap 43 forms an opening in the wall of the sheath 41, hence forming a slot 43 whose length extends from the distal end of the sheath 41 to a specified distance from the edge-lip 45 at the proximal end. The specified distance from the edge-lip 45 to the edge of the slot 43 keeps the trocar tip 2 covered when the sheath 41 is extended over the trocar 1. The specified distance ranges from 2 mm to 30 mm and is preferentially 10 mm in length. The slot 43 aligns with the position of the coupler guide rail 19 so that the sheath 41 and the coupler 18 each moves back and forth independently from and without interfering with the other's movement.

The top elevation view shows the top of the sheath 41 near the distal end comprises a sheath stop 48 (a “sheath catch-mechansim”) that stops the sheath 41 against the inner wall (a “handle catch-mechanism”) of the dispenser handle proximal aperture 9. This stop 48 prevents the sheath 41 from extending further out of the dispenser handle 7 when the sheath 41 is fully extended.

The side cutaway section N-N view shows the underside at the distal end of the sheath 41 comprises a sheath trigger release button 47 that, in the neutral position, locks the sheath 41 in the fully extended position out of the dispenser handle 7 so that the trocar 1 is fully and safely covered and protected (the “Disarmed Mode”). In the Disarmed Mode, the sheath trigger release button 47 fits within and protrudes out of the dispenser handle trigger release button opening 12. The sheath trigger release button 47 keeps the sheath in the extended position because the sheath trigger release button 47 is blocked from moving out of the dispenser handle opening 12 by the front and back-side walls of the opening 12.

When the sheath trigger release button 47 (a “sheath catch-release mechanism”) is depressed, the sheath 41 is released from the extended position and permitted to retract into the dispenser handle 7, thereby exposing the trocar tip 2 for deployment (the “Armed Mode”). The sheath trigger release button 47 is preferentially formed out of plastic that is attached to the sheath 41 at the proximal end and curved in the shape of a semicircle or other shape towards the distal end of the sheath 41. It is contemplated that alternative materials for the sheath trigger release button 47 comprise metal or other rigid materials of sufficient tensile strength to retain its formed shape and to return to its formed shape after being depressed.

When the sheath 41 is fully retracted into the dispenser handle 7, the sheath trigger release button 47 is back-stopped against the lip back-stop 14 inside the dispenser handle 7 by friction between the sheath trigger release button 47 and the lip back-stop 14 (as discussed in FIG. 7). This friction provides tactile resistance against extracting the sheath 41 out of the dispenser handle 7 and keeps the sheath 41 fully refracted, unless the clinician consciously exerts pulling force on the sheath 41 to re-extract the sheath 41 out of the handle 7. The above features notwithstanding, it is contemplated an alternative embodiment for the sheath 41 is to fabricate it without a trigger release button 47.

The side cutaway section N-N view of FIG. 14 also shows the sheath proximal end trocar tip support 46, which supports the encased trocar when the sheath is fully extended over the trocar. The diameter of the sheath proximal end trocar tip support 46 matches the diameter of the selected trocar 1, hence is chosen from 0.5 mm to 15 mm (plus a minimal manufacturing tolerance clearance) to fit the selected trocar 1. Therefore, the sheath proximal end trocar tip support 46 is customized and selected to fit a particular trocar. The above features notwithstanding, it is contemplated an alternative embodiment for the dispenser 6 is to fabricate it without a sheath 41.

FIG. 15 illustrates an exploded view of the preferred embodiment of the receiver 49 (a “handle grip”). The receiver 49 is comprised of a receiver handle 51, an integrated receiver coupler 50, an integrated locking mechanism 52 and a shield 53.

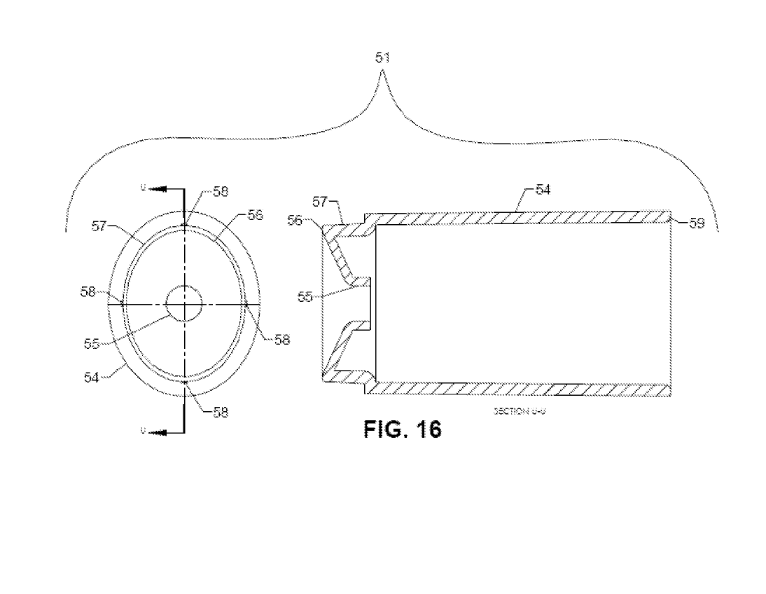

FIG. 16 illustrates a backwards-looking elevation view and side cutaway section U-U view of the preferred embodiment of the receiver handle 51. Generally, the receiver handle grip 54 is a rigid, hollow, cylindrical encasement with a receiver handle grip inner aperture 55 at the proximal end and a hollow interior through which a trocar tip 2 is inserted. Since the invention is designed to accommodate a trocar whose diameter may range from 0.5 mm to 15 mm, the diameter of the proximal receiver handle grip aperture 55 is chosen from a range of 0.5 mm to 15 mm (plus a minimal manufacturing tolerance clearance) to fit the selected trocar 1. Therefore, the proximal receiver handle grip aperture 55 is customized and selected to fit a particular trocar.

At the proximal end of the receiver handle grip 54, the outside diameter of the receiver handle grip joining-surface 57 is sized to match the inner surface of the shield distal aperture 111, preferentially 22 mm in width by 28.5 mm in height and formed in an elliptical shape, so that the shield 53 is fitted over and retained on the receiver handle grip 54. It is contemplated an alternative shape also comprises a circle, rectangle, or other shapes. Four protuberances 58 are formed on the receiver handle grip joining-surface 57 to provide frictional resistance when inserting the shield distal aperture 111 over the receiver handle grip joining-surface 57 so that a tight fit is achieved between the shield 53 and the receiver handle grip 54, and the shield 53 will not slide off without the application of significant twisting and pulling force.

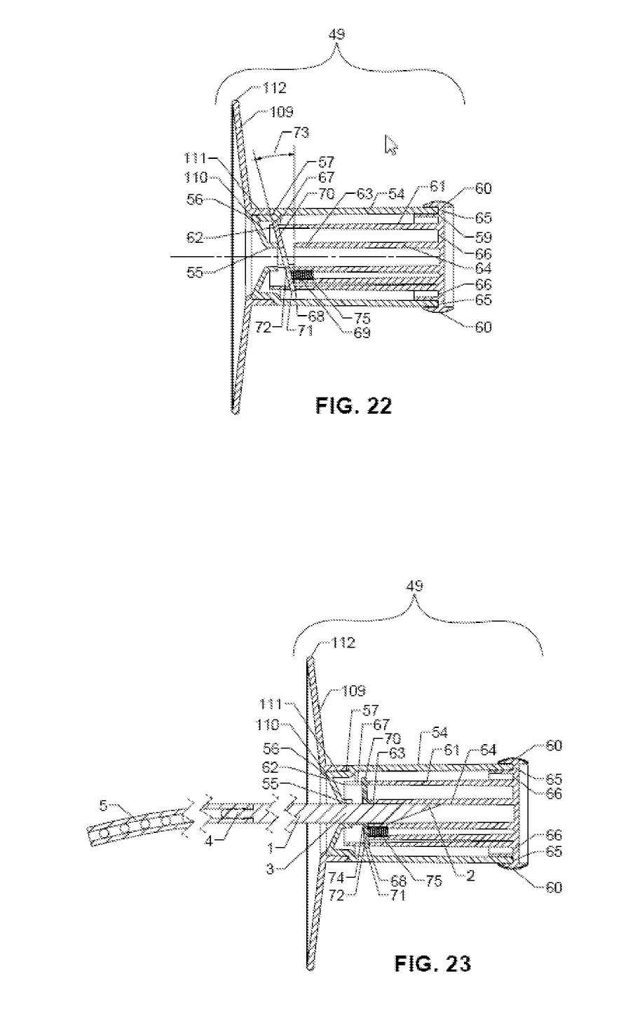

The diameter of the receiver handle grip outer aperture 56 at the proximal end of the receiver handle grip 54 is sized to match the shield proximal aperture 110, (discussed in FIG. 22, FIG. 39, and others) so that the shield proximal surface 109 mates smoothly and contiguously with the proximal surface of the receiver handle grip 54.

The outer dimensions of the receiver handle grip 54 are shaped preferentially as an ellipse and sized so that a clinician may comfortably hold the receiver handle grip 54 in his hand, preferentially 26 mm in height by 33 mm in width. It is contemplated an alternative shape also comprises a circle, rectangle, or other shapes. The receiver handle grip distal circumferential edge 59 is open so that it can be inserted and fixated into an adjoining slot 65 in the flared base 60 of the receiver coupler 50.

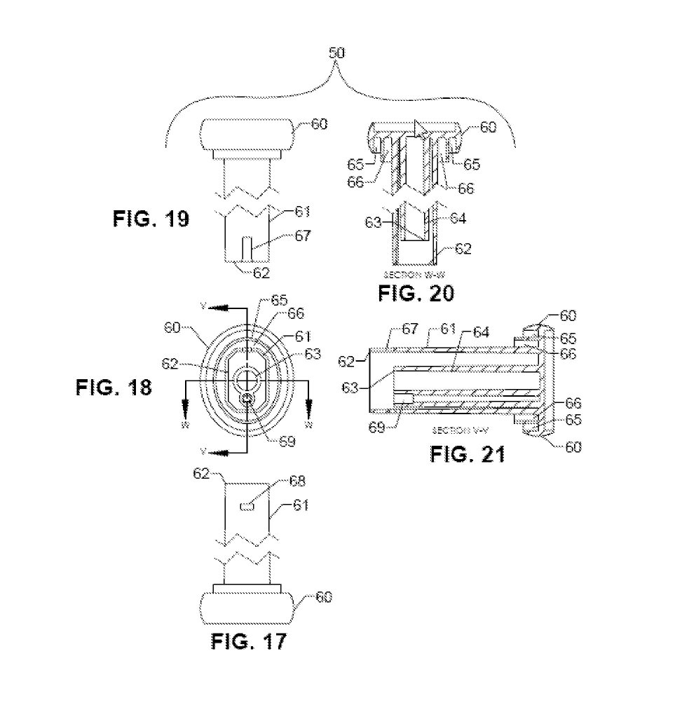

FIG. 17 through 21 illustrate different views of the preferred embodiment of the receiver coupler 50.

FIG. 17 shows a bottom elevation view; FIG. 18 shows a backwards-looking elevation view; FIG. 19 shows a top elevation view; FIG. 20 shows a bottom cutaway section W-W view; FIG. 21 shows a side cutaway section V-V view of the receiver coupler 50.

The bottom elevation view in FIG. 17 shows that the bottom surface at the proximal end of the receiver coupler outer surface 61 comprises a receiver coupler bottom slot 68 that is located a specified distance from the proximal edge of the receiver coupler 50 in the direction parallel to the axial channel and is preferentially 4.5 mm wide and 2 mm long. The specified distance for the receiver coupler bottom slot 68 from the proximal edge of the receiver coupler outer surface 61 ranges from 2 mm to 10 mm and preferentially is 9 mm.

The backwards-looking elevation view in FIG. 18 shows that generally, the receiver coupler 50 is a rigid, hollow, rectangular encasement 61 with bevel-shaped corners forming an outer aperture 62 of dimensions preferentially 14.5 mm by 21.5 mm, and a rounded flared base 60 of size preferentially 31 mm in width by 37 mm in height and formed in an elliptical shape. It is contemplated an alternative shape also comprises a circle, rectangle, or other shapes. This elevation view shows the receiver coupler inner aperture 63 through which a trocar tip 2 is inserted. Since the invention is designed to accommodate a trocar whose diameter may range from 0.5 mm to 15 mm, the diameter of the receiver coupler aperture 63 (and the adjoining receiver axial channel 64) is chosen from a range of 0.5 mm to 15 mm (plus a minimal manufacturing tolerance clearance) to fit the selected trocar 1. Therefore, the receiver coupler aperture 63 (and the adjoining receiver axial channel 64) is customized and selected to fit a particular trocar 1.

FIG. 18 shows the receiver coupler spring channel 69, located below the receiver coupler aperture 63. The diameter of the spring channel 69 is sized to house the locking mechanism spring 75 (discussed below).

FIG. 18 shows a circumferential slot 65 formed in the flared base 60. The inner space 66 is the hollow internal space between the sidewall of slot 65 and the sidewall of the receiver coupler axial channel 64.

The top elevation view in FIG. 19 shows that the top surface at the proximal end of the receiver coupler 61 comprises a gap 67 that is preferentially 3 mm wide and extends a specified length in the direction parallel to the axial channel. The gap 67 forms an opening in the surface of the receiver coupler 61, hence forms the receiver coupler top slot 67 that defines the amount of travel permitted for an adjoining locking plate top tab 70. The specified length for the receiver coupler top slot 67 ranges from 2 mm to 20 mm and preferentially is 8 mm.

The bottom cutaway section W-W view in FIG. 20 shows the receiver coupler inner aperture 63 and the receiver axial channel 64, through which a trocar tip 2 is inserted. The receiver coupler outer aperture 62 is open-ended to accept the insertion of the receiver handle grip inner aperture surface 55; the receiver coupler aperture 63 aligns to the receiver handle grip aperture 55, and the receiver coupler axial channel 64 aligns to the axial channel of the receiver handle grip 54.

FIG. 20 also shows the distal end of the receiver coupler 50 tapers slightly wider into a rounded flared base 60, to a diameter preferentially of 37 mm, to provide a comfortable backstop for the bottom of the clinician's gripping hand. The base at the distal and bottom end of the receiver coupler 50 is preferentially closed and solid. This figure also shows the circumferential slot 65 formed in the flared base 60 and the inner hollow space 66.

The side cutaway section V-V view in FIG. 21 is similar to the bottom cutaway section W-W view of FIG. 20 and shows the receiver coupler outer aperture 62, the receiver coupler inner aperture 63 at the proximal end of the receiver coupler 50, the receiver axial channel 64, the flared base 60, and the circumferential slot 65 formed in the flared base 60.

FIG. 21 also shows the receiver coupler spring channel 69 located below the receiver coupler axial channel 64, parallel to the direction of the axial channel. The spring channel 69 is positioned behind and adjacent, in the distal direction, to the position of a locking plate 72 (discussed below). The diameter of the spring channel 69 is sized to house the locking mechanism spring 75 (discussed below).

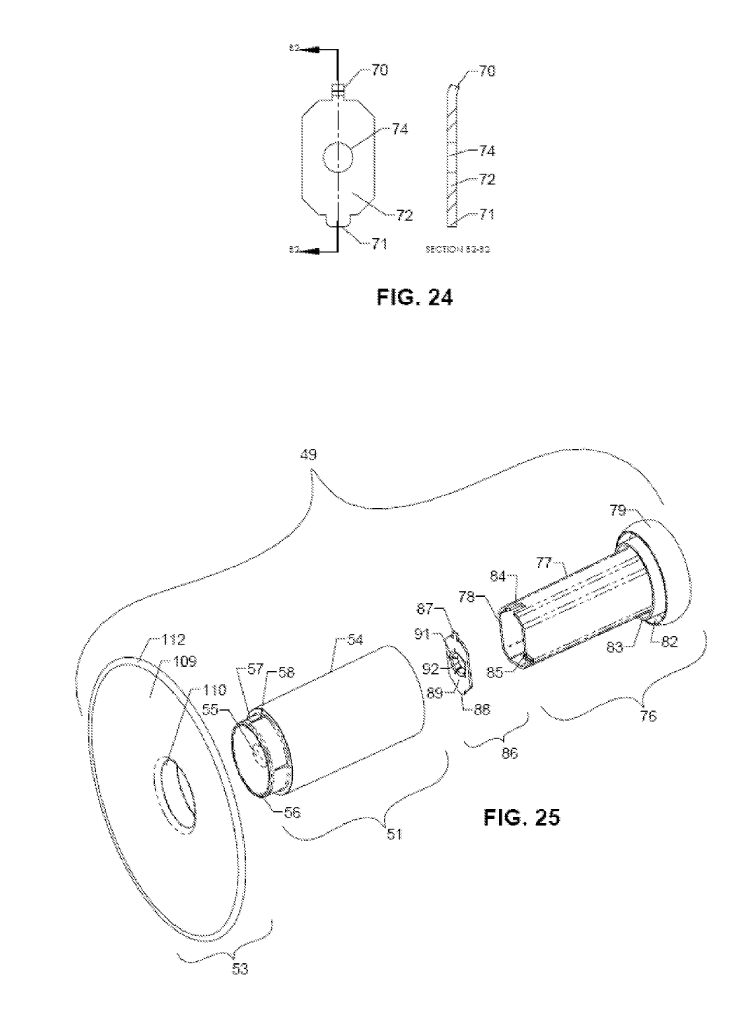

FIG. 22 shows a side cutaway view of the integrated receiver 49, comprised of the shield 53 including the shield surface 109 and other features discussed in FIG. 39, the receiver handle 51 including the receiver handle grip 54 and other features discussed in FIG. 16, the receiver coupler 50 including the receiver coupler outer surface 61 and other features discussed in FIGS. 17 to 21, and the locking mechanism 52 including the locking plate 72 and other features discussed in FIGS. 23 and 24. No trocar is present in the system in this figure.

This figure shows the attachment of the shield 53 to the receiver handle 51 via the shield distal aperture's 111 insertion onto the receiver handle grip joining-surface circumference 57 at the proximal outer edge of the receiver handle grip outer aperture 56. The surfaces are fixated via friction between the shield distal aperture inner surface 111 and the receiver handle grip joining-surface protuberances 58 as noted in FIG. 16. This figure also shows the contiguous surface at the junction between the shield proximal surface 109, shield proximal aperture 110, the receiver handle grip outer aperture 56 and the proximal surface of the receiver handle grip 54.

This figure shows the connection of the receiver coupler 50 to the receiver handle 51 via the concentric insertion of the receiver coupler outer surface 61 into the open-ended receiver handle grip 54. The receiver coupler outer surface 61 is inserted until the back-edge of the receiver handle grip distal circumferential edge 59 becomes inserted into the circumferential slot 65 formed in the flared base 60 of the receiver coupler 50. The receiver handle grip distal circumferential edge 59 is fixated into the receiver coupler circumferential slot 65 with an adhesive known in the art to bind plastic or metal, or is fixated simply by the friction of a tight fit. The receiver coupler inner aperture 63 aligns to the receiver handle grip aperture 55, and the receiver coupler axial channel 64 aligns within the hollow interior of the receiver handle grip 54.

This figure shows the preferred embodiment of an integrated locking mechanism 52 (a “locking mechanism”) comprised of a rigid locking plate 72 (a “rigid plate” or “plate”) and a spring 75 (discussed further in FIG. 24 below). A locking plate bottom tab 71 fits into the adjoining receiver coupler bottom slot 68 and a locking plate top tab 70 fits into and travels within the space defined by the adjoining receiver coupler top slot 67 and the distal edge of the receiver handle grip inner aperture 55. The combination of the position of the receiver coupler bottom slot 68, the length of the receiver coupler top slot 67, and the distal edge of the receiver handle grip inner aperture 55 defines an acute locking plate angle 73 for the plane containing the locking plate 72 relative to the direction parallel to the receiver coupler axial channel 64 in the proximal direction, measured at the point of the receiver coupler bottom slot 68. The range of specified distances and lengths defined in FIG. 19 and the thickness of the locking plate 72 defined in FIG. 24 result in a corresponding locking plate angle 73 ranging from 30 degrees to 90 degrees, preferentially 75 degrees.

This figure shows the locking mechanism spring 75 of the locking mechanism 52 is a compression spring with a diameter ranging from 2 mm to 10 mm, preferentially 5 mm, which fits inside the receiver coupler spring channel 69 and exerts unsupported compression force against the adjoining locking plate 72 in the proximal direction, pushing the locking plate 72 into locking plate angle 73. The spring 75 is composed of metal, plastic, or some other tensile material of sufficient tensile strength to pivot the plate within the receiver coupler 50.

FIG. 23 is similar to FIG. 22 and shows a side cutaway view of the integrated receiver 49, comprised of the shield 53 including the shield surface 109 and other features discussed in FIG. 39, the receiver handle 51 including the receiver handle grip 54 and other features discussed in FIG. 16, the receiver coupler 50 including the receiver coupler outer surface 61 and other features discussed in FIGS. 17 to 21, and the locking mechanism 52 including the locking plate 72 and other features discussed below and in FIG. 24. This figure shows the presence of a trocar 1 inserted into the receiver 49.

When inserting the trocar 1 into the receiver 49 in the distal direction, the trocar tip 2 passes through the concentrical shield proximal aperture 110, the receiver handle grip inner aperture 55, the locking plate aperture 74, the receiver coupler inner aperture 63, and into the receiver coupler axial channel 64. In the distal direction of insertion, the trocar shaft 3 pushes against the locking plate aperture 74 and thereby aligns the locking plate aperture 74 with the direction of the receiver coupler axial channel 64, therefore into a 90 degree locking plate angle 73 that permits the trocar 1 to pass through the aligned locking plate aperture 74 with low insertion force.

When attempting to extract the trocar 1 out of the receiver 49 in the proximal direction, the pulling force of the trocar shaft 3 against the locking plate aperture 74 no longer counterposes against the pushing force of the spring 75 but instead supplements the spring's 75 pushing force. Hence, the pushing force of the spring 75 circumvolves the locking plate 72 and locking plate top tab 70 (as permitted within the length of the receiver coupler top slot 67), rotating the locking plate 72 about the fixed point defined by the receiver coupler bottom slot 68 into an acute locking plate angle 73. The reduced apparent cross-section of the locking plate aperture 74 induced by the deflected locking plate 72 causes the sidewall of the trocar shaft 3 to rub and bind against the edge of the locking plate aperture 74, with friction that increases with the amount of pulling force exerted upon the trocar 1, hence firmly retaining the trocar 1 within the receiver 49.

FIG. 24 shows a backwards-looking elevation view and a side cross section B2-B2 view of the preferred embodiment of the locking plate 72 portion of the locking mechanism 52. The rigid locking plate 72 is formed in a rectangular shape with beveled edges to fit within the size and shape of the receiver coupler outside surface 61. The locking plate 72 comprises a locking plate bottom tab 71 and a locking plate top tab 70. The diameter of the locking plate aperture 74 (a “locking element aperture”) is chosen to match the diameter of the selected trocar (plus a minimal manufacturing tolerance clearance). Since the invention is designed to accommodate a trocar whose diameter may range from 0.5 mm to 15 mm, the diameter of the locking plate aperture 74 is chosen from a corresponding range of 0.5 mm to 15 mm (plus a minimal manufacturing tolerance clearance). Therefore, the locking plate 72 is customized and selected to fit a particular trocar 1. The rigid locking plate 72 is composed of metal, plastic, or some other rigid material known in the art.

FIG. 25 illustrates an exploded view of an alternative embodiment of the receiver 49. The receiver 49 is comprised of a receiver handle 51, an alternative embodiment of the integrated receiver coupler 76, an alternative embodiment of the integrated locking mechanism 86, and a shield 53.

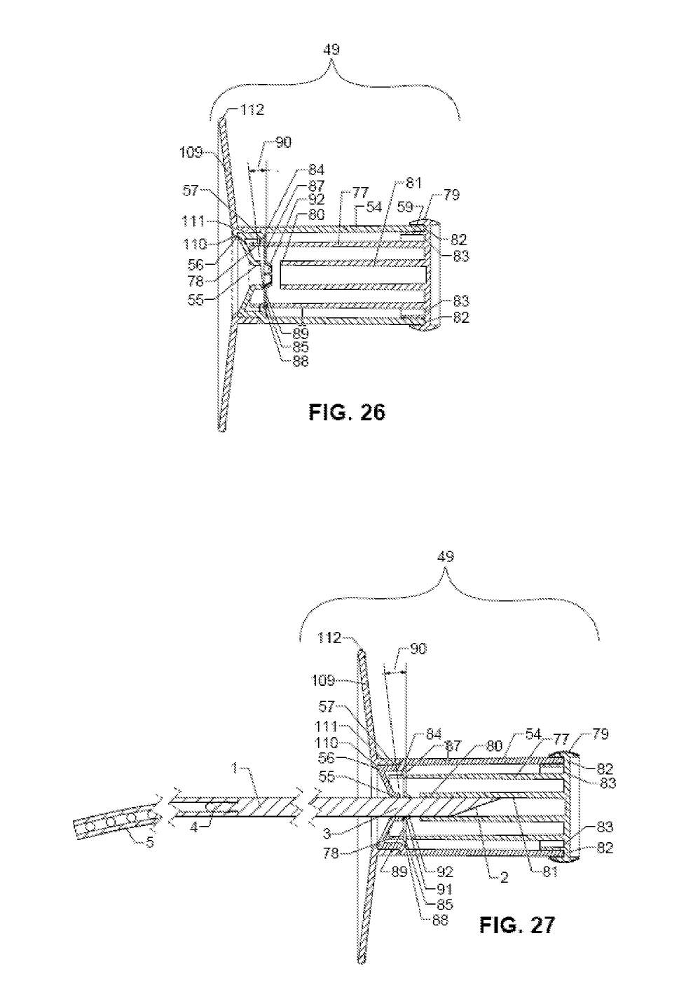

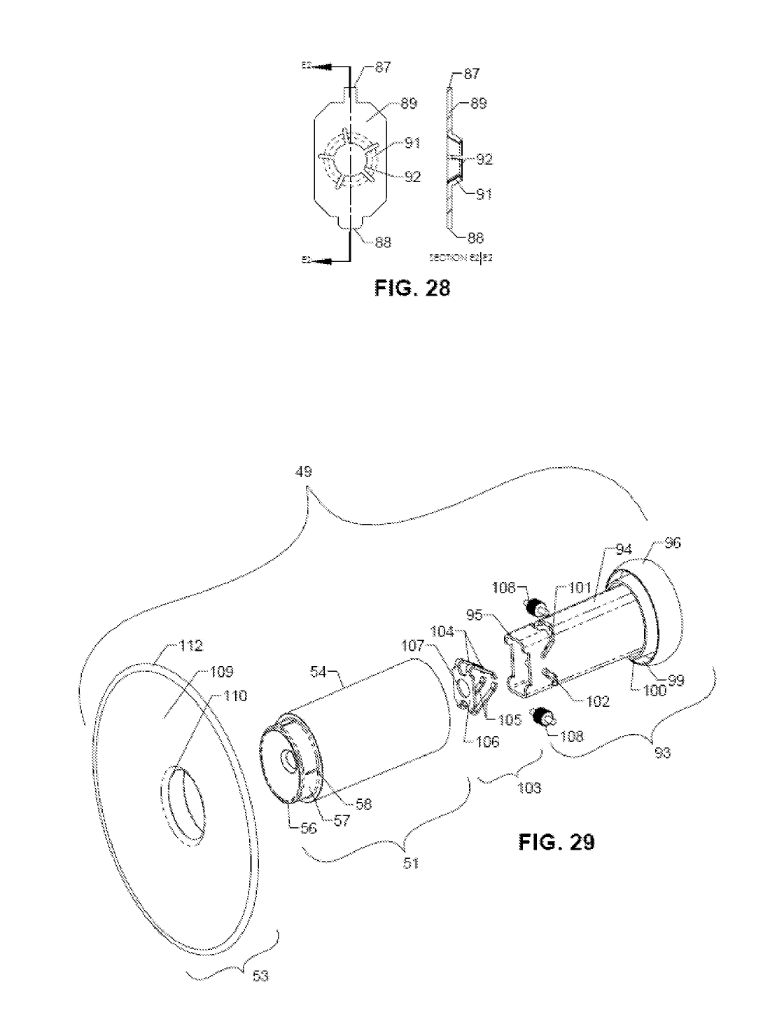

FIG. 26 shows a side cutaway view of the integrated receiver 49, comprised of the shield 53 including the shield surface 109 and other features discussed in FIG. 39, the receiver handle 51 including the receiver handle grip 54 and other features discussed in FIG. 16, the receiver coupler 76 including features discussed below, and the locking mechanism 86 including the locking plate 89 and other features discussed in FIG. 28. No trocar is present in the system in this figure.

Similar to FIG. 22, FIG. 26 shows the attachment of the shield 53 to the receiver handle 51 via the shield distal aperture 111 inserted onto the receiver handle grip joining-surface circumference 57 at the proximal outer edge of the receiver handle grip 54. The surfaces are fixated via friction between the inner surface of the shield distal aperture 111 and the receiver handle grip joining-surface protuberances 58 as noted in FIG. 16. This figure shows the contiguous surface at the junction between the shield proximal surface 109, shield proximal aperture 110, and the receiver handle grip outer aperture 56.

FIG. 26 shows the connection of the receiver coupler 76 to the receiver handle 51 via the concentric insertion of the receiver coupler outer surface 77 into the open-ended receiver handle grip 54. The receiver coupler outer surface 77 is inserted until the back-edge of the receiver handle grip distal circumferential edge 59 becomes inserted into the circumferential slot 82 formed in the flared base 79 of the receiver coupler 76. The receiver handle grip distal circumferential edge 59 is fixated into the receiver coupler circumferential slot 82 with an adhesive known in the art to bind plastic or metal, or is fixated simply by the friction of a tight fit. The receiver coupler inner aperture 80 aligns to the receiver handle grip aperture 55, and the receiver coupler axial channel 81 aligns to the hollow interior of the receiver handle grip 54.

This figure shows an alternative embodiment of an integrated locking mechanism 86 comprised of a rigid locking plate 89 (discussed further in FIG. 28 below. A locking plate bottom tab 88 fits into the adjoining receiver coupler bottom slot 85 and a locking plate top tab 87 fits into and travels within the space defined by the adjoining receiver coupler top slot 84 and the distal edge of the receiver handle grip aperture 55. The combination of the position of the receiver coupler bottom slot 85, the length of the receiver coupler top slot 84, and the distal edge of the receiver handle grip aperture 55 defines an acute locking plate angle 90 for the plane containing the locking plate 89 relative to the direction parallel to the receiver coupler axial channel 81 in the proximal direction, measured at the point of the receiver coupler bottom slot 85. The range of specified distances and lengths defined for the receiver coupler top slot 84 (similarly to as discussed in FIG. 19) and the thickness of the locking plate 89 defined in FIG. 28 result in a corresponding locking plate angle 90 ranging from 30 degrees to 90 degrees, preferentially 75 degrees. Another alternative embodiment fixes the receiver coupler top slot 84 in a single position opposite from the receiver coupler bottom slot 85, resulting in a fixed locking plate angle 90 of 90 degrees.

FIG. 27 is similar to FIG. 26 and shows a side cutaway view of the integrated receiver 49, comprised of the shield 53 including the shield surface 109 and other features discussed in FIG. 39, the receiver handle 51 including the receiver handle grip 54 and other features discussed in FIG. 16, the alternative embodiment of the receiver coupler 76 including features discussed below, and the alternative embodiment of the locking mechanism 86 including the locking plate 89 and other features discussed below and in FIG. 28. This figure shows the presence of a trocar 1 inserted into the receiver 49.

When inserting the trocar 1 into the receiver 49 in the distal direction, the trocar tip 2 passes through the concentrical shield proximal aperture 110, the receiver handle grip inner aperture 55, the locking plate aperture 91, the receiver coupler inner aperture 80, and into the receiver coupler axial channel 81. In the distal direction of insertion, the trocar shaft 3 pushes through the locking plate aperture 91 in the same direction as the locking plate aperture protuberances 92 (discussed in FIG. 28) are directed, so that insignificant resistance is imposed on the trocar shaft 3 by the locking plate aperture protuberances 92. Moreover, the trocar shaft 3 pushes against the locking plate aperture 91 and aligns the locking plate aperture 91 with the direction of the receiver coupler axial channel 81, therefore into a 90 degree locking plate angle 90 that permits the trocar 1 to pass through the aligned locking plate aperture 91 with low insertion force.

When attempting to extract the trocar 1 out of the receiver 49 in the proximal direction, the direction of the pulling force of the trocar shaft 3 opposes the direction that the locking plate aperture protuberances 92 are directed towards so that the protuberances 92 impose significant frictional force against the trocar shaft 3. Moreover, the friction locking plate aperture 91 and the trocar shaft 3 circumvolves the locking plate 89 and locking plate top tab 87 (as permitted within the length of the receiver coupler top slot 84), rotating the locking plate 89 about the fixed point defined by the receiver coupler bottom slot 85 into an acute locking plate angle 90. Therefore, in addition to the frictional opposing force imposed by the locking plate aperture protuberances 92 against the trocar shaft 3, the reduced apparent cross-section of the locking plate aperture 91 induced by the deflected locking plate 89 causes the sidewall of the trocar shaft 3 to rub and bind against the edge of the locking plate aperture protuberances 92 and locking plate aperture 91, with friction that increases with the amount of pulling force exerted upon the trocar 1, hence firmly retaining the trocar 1 within the receiver 49.

FIG. 28 shows a backwards-looking elevation view and a side cross section E2-E2 view of the alternative embodiment of the locking plate 89 portion of the locking mechanism 86. The rigid locking plate 89 is formed in a rectangular shape with beveled edges and fabricated to fit within the size and shape of the receiver coupler outside surface 77. The locking plate 89 comprises a locking plate bottom tab 88 formed to fit within the receiver coupler bottom slot 85 and a locking plate top tab 87 formed to fit within the receiver coupler top slot 84. The diameter of the locking plate aperture 91 is customized to match the diameter of the selected trocar. Locking plate aperture protuberances 92 are formed around the circumference of the locking plate aperture 91, which are preferentially 2 mm in length, crimped in the distal direction at an approximately 45 degree angle measured from the direction transverse to the axial direction and towards the distal direction, and extend in the distal direction. Since the invention is designed to accommodate a trocar whose diameter may range from 0.5 mm to 15 mm, the diameter of the locking plate aperture 91 is chosen from a range of 0.5 mm-15 mm (plus any minimal manufacturing tolerance clearances). Therefore, the locking plate 89 is customized and selected to fit a particular trocar. The rigid locking plate 89 is composed of metal, plastic, or some other rigid material known in the art, provided that the tensile strength of the locking plate aperture protuberances 92 is sufficient to retain its position after crimping and when resisting the friction force of a trocar's 1 insertion or retraction.

FIG. 29 illustrates an exploded view of a second alternative embodiment of the receiver 49. The receiver 49 is comprised of a receiver handle 51, a second alternative embodiment of the integrated receiver coupler 93, a second alternative embodiment of the integrated locking mechanism 103, and a shield 53.

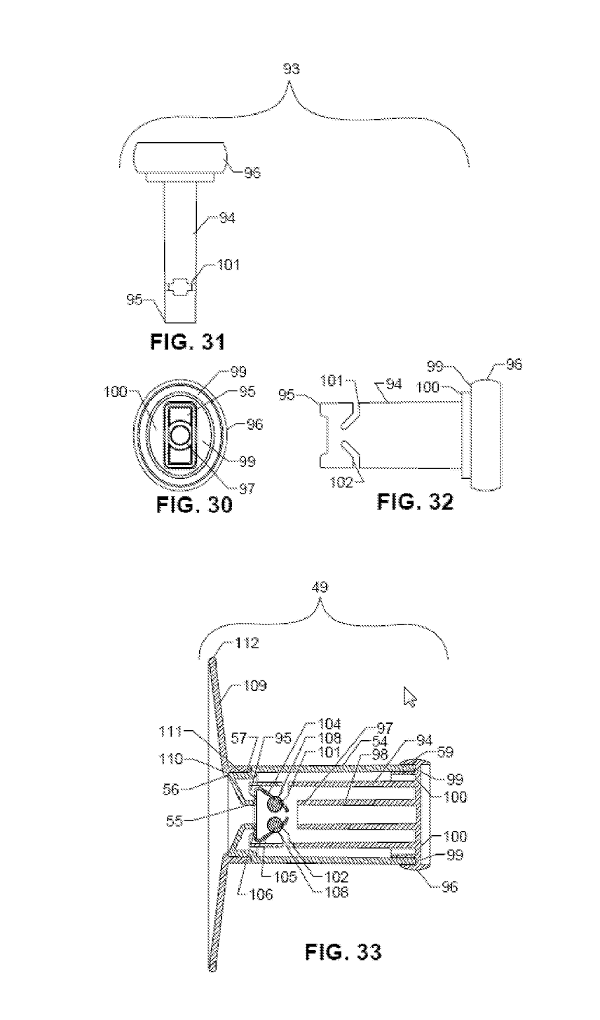

FIG. 30 through 32 illustrate different views of the second alternative embodiment of the receiver coupler 93. FIG. 30 shows a backwards-looking elevation view; FIG. 31 shows a top elevation view; FIG. 32 shows a side elevation view.

The backwards-looking elevation view in FIG. 30 shows that generally, the receiver coupler 93 is a rigid, hollow, rectangular encasement 94 with rounded-shaped corners forming an outer aperture 95, of dimensions preferentially 14.5 mm by 21.5 mm, and a rounded flared base 96 of size 31 mm in width by 37 mm in height formed in an elliptical shape. It is contemplated an alternative shape also comprises a circle, rectangle, or other shapes. This elevation view shows the receiver coupler inner aperture 97 through which a trocar tip 2 is inserted. Since the invention is designed to accommodate a trocar 1 whose diameter may range from 0.5 mm to 15 mm, the diameter of the receiver coupler aperture 97 (and the adjoining receiver axial channel 98) is chosen from a range of 0.5 mm-15 mm (plus a minimal manufacturing tolerance clearance) to fit the selected trocar 1. Therefore, the receiver coupler aperture 97 (and the adjoining receiver axial channel 98) is customized and selected to fit a particular trocar.

FIG. 30 shows a circumferential slot 99 formed in the flared base 96. The inner space 100 is the hollow internal space between the sidewall of slot 99 and the sidewall of the receiver coupler inner aperture 97 and receiver coupler axial channel 98.

The top elevation view in FIG. 31 shows that the top surface at the proximal end of the receiver coupler 93 comprises a receiver coupler top slot 101 that is formed by a cut-out hole located a specified distance from the proximal edge of the receiver coupler 93 in the direction parallel to the receiver coupler axial channel 98 and is preferentially 2.25 mm wide by 4.3 mm long, and a groove across the top of the receiver coupler 93 that bisects the cut-out hole in the direction transverse to the receiver coupler axial channel 98, which is preferentially 2.25 mm in width. The receiver coupler top slot 101 is mirrored identically to the bottom of the receiver coupler 93 and forms the receiver coupler bottom slot 102. The receiver coupler top slot 101 and receiver coupler bottom slot 102 each form an opening through which the locking rollers 108 are inserted into the receiver coupler 93.

The side elevation view in FIG. 32 shows the receiver coupler outer aperture 95, which is open-ended to accept the insertion of the receiver handle grip inner aperture surface 55. This figure shows the receiver coupler top slot 101 and bottom slot 102, illustrating the slot cutting inwards in the direction transverse to the receiver coupler axial channel 98, preferentially a length of 2.25 mm, and a bend in the slots from the direction transverse to the receiver coupler axial channel 98 in the proximal direction, preferentially 45 degrees (measured from the direction transverse to the receiver coupler axial channel 98 and towards the proximal direction), of length preferentially 6 mm.

This figure shows the distal end of the receiver coupler 93 tapers slightly wider into a rounded flared base 96, of size preferentially 31 mm in width by 37 mm in height and formed in an elliptical shape, to provide a comfortable backstop for the bottom of the clinician's gripping hand. It is contemplated an alternative shape also comprises a circle, rectangle, or other shapes. The base at the distal and bottom end of the receiver coupler 93 is preferentially closed and solid. This figure also shows the circumferential slot 99 formed in the flared base 96 and the inner hollow space 100.

FIG. 33 shows a side cutaway view of the integrated receiver 49, comprised of the shield 53 including the shield surface 109 and other features discussed in FIG. 39, the receiver handle 51 including the receiver handle grip 54 and other features discussed in FIG. 16, the receiver coupler 93 including features discussed below, and the locking mechanism 103 including the locking plate 106 and locking rollers 108 discussed in FIGS. 35 to 38. No trocar is present in the system in this figure.

Similar to FIG. 22, FIG. 33 shows the attachment of the shield 53 to the receiver handle 51 via the shield distal aperture 111 inserted onto the receiver handle grip joining-surface circumference 57 at the proximal outer edge of the receiver handle grip 54. The surfaces are fixated via friction between the shield distal aperture surface 111 and the receiver handle grip joining-surface protuberances 58 as noted in FIG. 16. This figure shows the contiguous surface at the junction between the shield proximal surface 109, shield proximal aperture 110, and the receiver handle grip outer aperture 56.

This figure shows the connection of the receiver coupler 93 to the receiver handle 51 via the concentric insertion of the receiver coupler outer surface 94 into the open-ended receiver handle grip 54. The receiver coupler outer surface 94 is inserted until the back-edge of the receiver handle grip distal circumferential edge 59 becomes inserted into the circumferential slot 99 formed in the flared base 96 of the receiver coupler 93. The receiver handle grip distal circumferential edge 59 is fixated into the receiver coupler circumferential slot 99 with an adhesive known in the art to bind plastic or metal, or is fixated simply by the friction of a tight fit. The receiver coupler inner aperture 97 aligns to the receiver handle grip aperture 55, and the receiver coupler axial channel 98 aligns within the axial channel of the receiver handle grip 54.

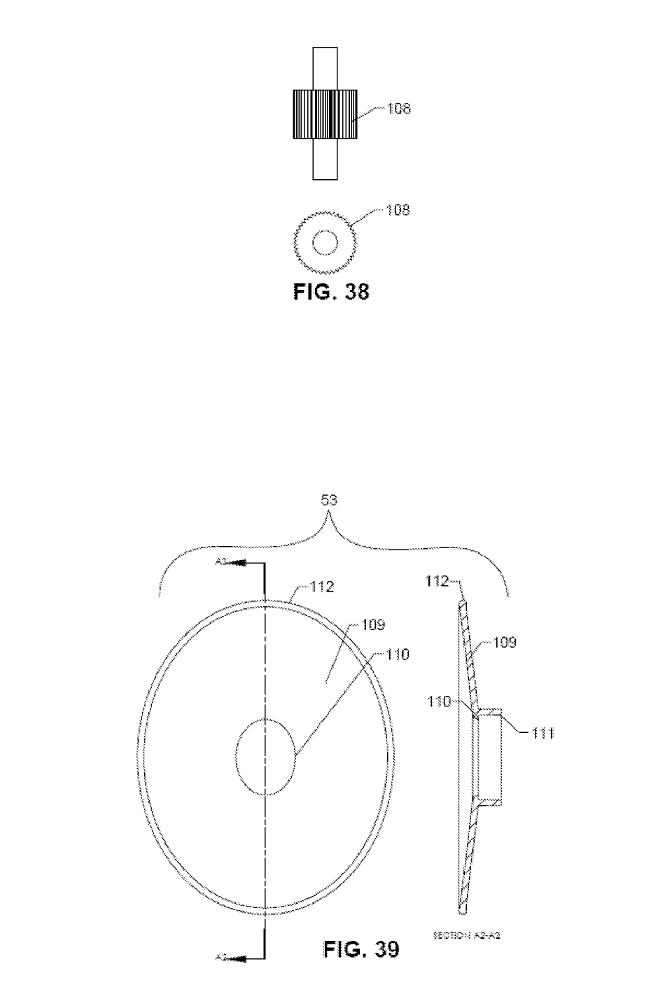

This figure shows the second alternative embodiment of an integrated locking mechanism 103 comprising a rigid locking plate 106 (discussed in FIG. 37) and a top and bottom locking roller 108 (discussed in FIG. 38). Locking plate bottom bracket leads 105 (a “clip”) extend in the distal direction and align with the adjoining receiver coupler bottom slot 102 and locking plate top bracket leads 104 (a “clip”) extend in the distal direction and align with the adjoining receiver coupler top slot 101. The axles of a bottom and top locking roller 108 lie within the receiver coupler bottom slot 102 and the receiver coupler top slot 101, respectively, and are pushed inwards (towards the center of the axial channel) by the tensile force of the locking plate bottom bracket leads 105 and locking plate top bracket leads 104, respectively.

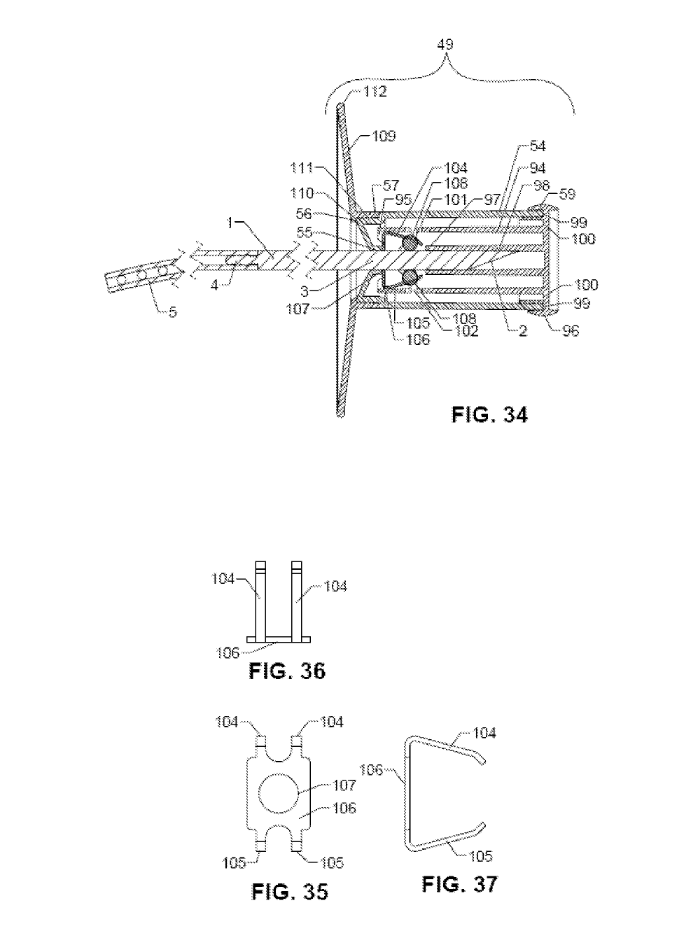

FIG. 34 is similar to FIG. 33 and shows a side cutaway view of the integrated receiver 49, comprised of the shield 53 including the shield surface 109 and other features discussed in FIG. 39, the receiver handle 51 including the receiver handle grip 54 and other features discussed in FIG. 16, the second alternative embodiment of the receiver coupler 93 including features discussed below, and the second alternative embodiment of the locking mechanism 103 including the locking plate 106 and the locking rollers 108 discussed below and in FIGS. 37 and 38. This figure shows the presence of a trocar 1 inserted into the receiver 49.

When inserting the trocar 1 into the receiver 49 in the distal direction, the trocar tip 2 passes through the concentrical shield proximal aperture 110, the receiver handle grip inner aperture 55, the locking plate aperture 107, the receiver coupler inner aperture 97, and into the receiver coupler axial channel 98. In the distal direction of insertion, the trocar shaft 3 pushes through the locking plate aperture 107 and through the locking rollers 108, pushing the locking rollers 108 outwards in their slots 101 and 102 in the distal direction, and away from the trocar shaft 3, which permits the trocar 1 to pass through the locking plate aperture 107 and locking rollers 108 with low insertion force.

When attempting to extract the trocar 1 out of the receiver 49 in the proximal direction, the pulling force of the trocar shaft 3 runs against the direction of the locking rollers 108 so that the rollers 108 are pulled in the proximal direction, hence inwards in their slots 101 and 102 and inwards towards the center of the receiver coupler axial channel 98. The compression of the locking rollers 108 towards and against the trocar shaft 3 induces friction that increases with the amount of pulling force exerted on the trocar 1, hence firmly retaining the trocar 1 within the receiver 49.

FIGS. 35 to 37 show a backwards-looking elevation view, a top elevation view, and a side elevation view of the second alternative embodiment of the locking plate 106 portion of the locking mechanism 103. The rigid locking plate 106 is formed in a rectangular shape with rounded edges and bracket leads at the bottom and top, fabricated to fit within the size and shape of the receiver coupler outside surface 94. The outside edges the rigid locking plate 106 are formed to lie against the adjoining edges of the receiver coupler outer aperture 95, which positions the rigid locking plate 106 against the proximal edges of the receiver coupler 93. The locking plate bottom bracket leads 105 are bent into an elbow at the bottom in the distal direction at an angle of preferentially 95 degrees, measured from the direction transverse to the receiver coupler axial channel 98 of the locking plate 106 and towards the distal direction. The locking plate top bracket leads 104 are similarly bent into an elbow in a mirrored fashion from bottom bracket leads 105. Preferentially, 2 mm from the distal ends of the locking plate bottom bracket leads 105 and locking plate top bracket leads 104 are formed a second bend comprising 45 degrees, measured from the direction of extension of the bracket lead and towards the center of the receiver coupler axial channel 98. This second bend helps retain the locking plate rollers 108 within their respective slots 101, 102 at the distal ends of the slots 101, 102. The locking plate 106 is composed of metal or rigid plastic so that, when the bracket leads 105 and 104 are formed into position, the leads 105 and 104 have sufficient tensile strength to hold their shape and push the axles of the locking rollers 108 inwards and towards the receiver coupler axial channel 98.

The diameter of the locking plate aperture 107 is chosen to match the diameter of the selected trocar 1 (plus a minimal manufacturing tolerance clearance). Since the invention is designed to accommodate a trocar 1 whose diameter may range from 0.5 mm to 15 mm, the diameter of the locking plate aperture 107 is chosen from a range of 0.5 mm to 15 mm (plus any minimal manufacturing tolerance clearances. Therefore, the locking plate 106 is customized and selected to fit a particular trocar.

FIG. 38 shows a backwards-looking elevation view and a top elevation view of the locking roller 108. The locking roller 108 is a wheel having a diameter preferentially of 5.5 mm, a center axle having a diameter preferentially of 2 mm, and fashioned with ridges along the outer circumference whose ribbings run parallel to the direction of the axial channel of the locking roller 108. The wheel and ridges are cast of metal or injection-molded in plastic chosen from the known art that is sufficiently hard to hold the edges when imposing friction against the pulling force of a trocar 1. Alternatively, the wheel and axle are injection-molded in a rigid plastic known in the art and a rubber o-ring is inserted over the wheel to impose friction against the pulling force of a trocar 1.

FIG. 39 shows a backwards-looking elevation view and a side cutaway section A2-A2 view of the preferred embodiment of the shield 53. Generally, the removable shield 53 is a rigid, circular disk (a “rigid shield” or “shield”) that is concave inwards in the distal direction, similar to a funnel, comprising a shield proximal surface 109, a shield proximal aperture 110, a distal aperture 111 and axial channel. The backwards-elevation view shows the shield lip 112, the shield proximal surface 109, and the shield proximal aperture 110.

The side cutaway section A2-A2 view shows the shield distal aperture 111. The preferred embodiment for connecting the inner surface of the shield distal aperture 111 to the receiver handle grip joining-surface 57 is by friction between the inner surface of the shield distal aperture 111 and the receiver handle grip joining-surface protuberances 58. Alternative embodiments for joining these surfaces include a tongue-in-groove feature formed on surface of the shield distal aperture 111 that snaps together or apart from corresponding features on the proximal end of the receiver handle grip joining-surface 57, or a blade feature formed on the shield distal aperture 111 that screws or twists to connect to or release from corresponding features on the receiver handle grip joining-surface 57. Another alternative embodiment for joining these surfaces includes applying an adhesive known in the art to bind plastics so that the shield 53 is permanently affixed to the receiver handle grip 54. When the shield 53 is attached to the receiver handle grip joining-surface 57, the shield aperture 110 aligns to the receiver handle grip inner aperture 55, to the axial channel of the receiver handle grip 54, to the locking plate aperture 74, to the receiver coupler inner aperture 63, and to the receiver coupler axial channel 64.

The degree of concavity of the shield proximal surface 109 disk ranges from 0 degrees to 60 degrees, preferentially 30 degrees, measured from the plane consisting of the circumferential edge of the shield 112 and towards the distal direction parallel to the axial channel. The diameter of the shield edge 112 is substantially wide enough to cover and protect the clinician's hand that grips the receiver handle 51 behind the shield, ranging from a diameter of 10 mm to 250 mm, preferentially 100 mm.

The preferred embodiment of the shield 53 includes a raised edge or lip 112 (a “raised rim”) around the outside circumference of the shield 53 in the proximal direction with height that ranges from 0.5 mm to 10 mm, preferentially 2 mm. The shield lip 112 acts as a guide-stop to prevent the trocar tip 2 from sliding off the shield 53 in the outward direction

Preferentially, an injection molded process is used to fabricate all parts of the invention, using a rigid plastic formulation known in the art, except for the parts as noted. It is also contemplated that all parts of the invention could alternatively be cast in metal or fabricated in another rigid material known in the art, except for the parts as noted.

### Method of Use

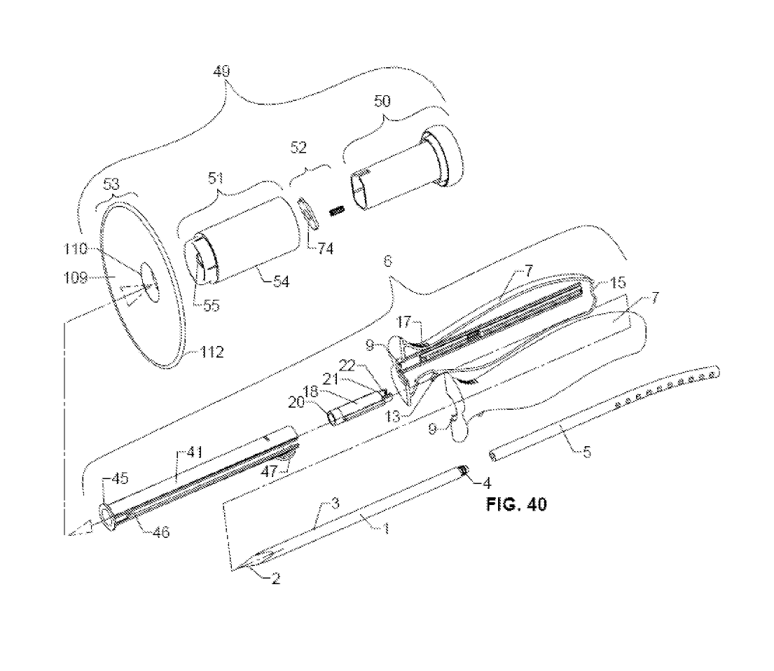

Referring to FIG. 40, the best mode of applying the Trocar Dispenser and Grip Receiver Safety System utilizes the preferred embodiment of the dispenser handle 7 and receiver 49, coupled with a trocar 1 of desired type and diameter, connected to drainage tubing 5 of desired length and type. The Trocar Dispenser and Grip Receiver Safety System comprises certain components that universally accommodate multiple trocar sizes and types, and certain custom-manufactured components that are customized and selected depending on the configuration and diameter of the trocar 1. The custom-manufactured components consist of the coupler 18, the sheath 41, the receiver handle 51, the receiver coupler 50, and the locking mechanism 52. For example, a 200 FR 100 mm long trocar with standard ribbed distal connector would dictate 100 mm diameter (plus a manufacturing tolerance clearance) is selected for the diameters of the coupler aperture 20, the sheath proximal end trocar tip support 46, the receiver handle grip inner aperture 55, the receiver coupler inner aperture 63, and the locking plate aperture 74.

The shield 53 is attached to the receiver hand grip 54, which is integrated with the receiver coupler 50 and the internal locking mechanism 52. The dispenser handle 7 is assembled so that the coupler 18 is axially aligned inside the sheath 41, which is axially aligned inside the dispenser handle 7. The drainage tubing 5 is connected to the trocar distal end 4 in the normal fashion. The trocar 1 is inserted into the coupler 18 and the drainage tubing 5 is threaded out the rear of the coupler distal aperture 21 and the dispenser handle distal aperture 15. The trocar 1 and drainage tubing 5 is seated in the dispenser handle 7 to engage the coupler gripping mechanism 22 onto the trocar distal end 4, preventing any further backwards movement by the trocar 1 into the dispenser handle 7. The sheath 41 is fully extended over the trocar tip 2, and the trigger release button 47 is engaged so the dispenser handle 7 is in the Disarmed Mode.

The clinician grips the dispenser handle 7 in one hand, positioning his thumb over the dispenser handle thumb grip 17. The clinician positions a finger on the dispenser handle trigger guard 13 to avoid inadvertently depressing the trigger release button 47. In this manner, the clinician directs the sheath 41 into the surgical cavity and manipulates the proximal end of the sheath 41 within the lumen and around any organs or tissue, without fear of inadvertently piercing any tissues with the protected trocar tip 2.

With his other hand, the clinician holds the receiver 49 and positions it on the outside of the surgical site to serve as a counterpressure and a target for the dispenser handle 7 and sheath 41. The receiver handle grip 54 provides a comfortable grip, while the large shield 53 permits the clinician to easily approximate a target for directing the dispenser 6 and protected trocar 1 as well as to provide tactile counterpressure against the patient's skin when the clinician pushes the trocar 1 through the skin.

When the clinician has positioned the sheath 41 in the lumen and pressed the sheath lip edge 45 against the patient's skin at the desired location, and positioned the receiver 49 against the patient's skin opposite the sheath 41, the clinician is ready to deploy the trocar 1. While maintaining pressure against the counterpressure, the clinician depresses the trigger release button 47 to disengage the sheath 41 and increases pressure slightly to push the trocar 1 and create a wound opening. Since the trigger release button 47 is depressed, the sheath 41 can slide backwards into the dispenser handle 7 (now in Armed Mode) and expose the trocar tip 2. As the clinician pushes the trocar 1 forward against and through the cavity wall and the patient's skin, the sheath 41 is pushed by the cavity wall and retracted into the dispenser handle 7.

Holding the receiver 49 with his other hand, the clinician presents the large shield 53 of the receiver 49 to accept the trocar tip 2 now emerging on the outside of the lumen. With his first hand, the clinician continues to push the trocar 1 all the way through the wound opening and adjusts his other hand with the receiver 49, if needed, to direct the trocar tip 2 into the shield's center aperture 110 and then into the receiver's locking mechanism 52. Because the diameter of the shield 53 completely covers the clinician's hand with ample overlap, the clinician is assured of protection from an inadvertent injury by the trocar tip 2. The concavity of the shield surface 109 encourages the tip 2 towards the center, and the edge lip 112 around the outer circumference of the shield 53 prevents the tip 2 from slipping off the shield in the case of an errant outwards-bound tip 2.

After pushing the trocar 1 into the receiver's locking mechanism 52, the locking mechanism 52 traps the trocar shaft 3 within the receiver 49. The clinician then withdraws the receiver 49 and locked trocar 1 away from the surgical site, extricating a segment of trailing drainage tubing 5 through the dispenser handle 7 and through the wound opening.

Within the dispenser handle 7, the coupler 18 no longer grips the trocar 1 since the trocar 1 (and drainage tubing 5) pulls forward (in the proximal direction). Although the coupler 18 stops within the dispenser handle at the extent of its permitted range of motion, the trocar 1 and tubing 5 continue moving forwards and out of the coupler aperture 20 and the dispenser handle aperture 9 in the proximal direction.

After extracting a segment of drainage tubing 5 from the wound opening, the clinician cuts the tubing 5 near the trocar distal end 4 and attaches the severed tubing 5 to an appropriate collection bag or reservoir. The clinician detaches the shield 53 from the receiver 49 and discards the shield 53 into a contaminated waste receptacle. The clinician discards the receiver 49 and the trocar 1 (safely locked within) into a contaminated sharps waste receptacle.

Within the surgical cavity, the clinician retrieves the dispenser 6 by threading the distal end of the remaining drainage tubing 5 through the dispenser 6. The clinician positions the distal end of the drainage tubing 5 as needed within the surgical cavity. The clinician then discards the dispenser 6 into a contaminated waste receptacle.

We claim:

1. A kit comprising:
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath; and
wherein the proximal end of said sheath is formed with a second sheath catch-mechanism on the outer surface of said sheath;
a handle comprising a rigid encasement with an axial handle channel that releasably encloses the shaft of said sheath and a trocar;
wherein the inner diameter of said handle channel substantially matches the outer diameter of said sheath, permitting said sheath to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient;
wherein the inner surface of the proximal end of said handle is formed with a first handle catch-mechanism that is adapted to interact with said first sheath catch-mechanism;
wherein the transverse edge at the proximal end of said handle is formed with a second handle catch-mechanism that is adapted to interact with said second sheath catch-mechanism;
wherein said first handle catch-mechanism is adapted to releasably stop said sheath in a substantially extended position when said first sheath catch-mechanism adjoins said first handle catch-mechanism;
wherein said second handle catch-mechanism is adapted to prevent said sheath from retracting into said handle channel when said second sheath catch-mechanism adjoins said second handle-catch mechanism; and
wherein the distal end of said handle channel is formed with a backstop that is adapted to prevent a trocar from retracting into the handle in the reverse direction, but adapted to permit the forward (proximal) direction of a trocar when extracting said trocar out of said handle and said sheath.

2. A kit comprising:
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath; and
wherein the proximal end of said sheath is formed with a second sheath catch-mechanism on the outer surface of said sheath;
a handle comprising a rigid encasement with an axial handle channel that releasably encloses the shaft of said sheath and a trocar;
wherein the inner diameter of said handle channel substantially matches the outer diameter of said sheath, permitting said sheath to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient;
wherein the inner surface of the proximal end of said handle is formed with a first handle catch-mechanism that is adapted to interact with said first sheath catch-mechanism;
wherein the transverse surface at the proximal end of said handle is formed with a second handle catch-mechanism that is adapted to interact with said second sheath catch-mechanism;
wherein said first handle catch-mechanism is adapted to releasably stop said sheath in a substantially extended position when said first sheath catch-mechanism adjoins said first handle catch-mechanism; and
wherein said second handle catch-mechanism is adapted to prevent said sheath from retracting into said handle channel when said second sheath catch-mechanism adjoins said second handle-catch mechanism;
a pathway within said handle channel in the distal direction is adapted to direct a trocar in a substantially divergent direction relative to said handle channel;
wherein said pathway is adapted to permit said trocar to slide backwards into a substantially retracted position within the handle and to be stopped in the retracted position when the distal end of said trocar has substantially reached the position of said divergent pathway in the distal direction, such that further travel by said trocar in the reverse (distal) direction is substantially blocked by the divergent direction of said divergent pathway in the distal direction; and
wherein said pathway is adapted to permit said trocar to slide forwards and out of said handle when extracting said trocar out of said handle in the proximal direction.

3. A kit comprising:
a coupler comprising a rigid encasement with an axial channel that releasably encloses the shaft of a trocar;
wherein the inner diameter of said channel substantially matches the outer diameter of said trocar shaft;
wherein said coupler is formed with feet adapted to exert inwards pressure and grip adjoining geometries formed in the shaft of a trocar when said trocar is pushed in the backwards direction into the coupler, and adapted to permit said trocar to slide in the forward direction through and out of the coupler, thereby releasably securing a trocar to said coupler in the backwards direction; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient;
wherein the inner surface of the distal end of said handle is formed with a first handle catch-mechanism that is adapted to interact with said coupler catch-mechanism;
wherein the inner surface of the proximal end of said handle is formed with a second handle catch-mechanism that is adapted to interact with said coupler catch-mechanism;
wherein said handle channel is adapted to permit said coupler to slide backwards into a substantially retracted position within said handle and adapted to releasably stop in the retracted position when said first handle catch-mechanism adjoins said coupler catch-mechanism and adapted to prevent said coupler and a trocar from retracting further beyond said first handle catch-mechanism into the handle in the reverse (distal) direction; and
wherein said handle channel is adapted to permit said coupler to slide forwards while not impeding the forward direction of said coupler and trocar when extracting said coupler and trocar out of said handle in the proximal direction, until said coupler catch-mechanism adjoins the second handle-catch mechanism at which point said second handle catch-mechanism is adapted to prevent said coupler from further sliding in the proximal direction out of said handle.

4. A kit according to claim 3, further comprising:
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a third handle catch-mechanism that is adapted to interact with said first sheath catch-mechanism;
wherein the transverse surface at the proximal end of said handle is formed with a fourth handle catch-mechanism that is adapted to interact with said second sheath catch-mechanism;
wherein said handle channel is adapted to stop said sheath in a substantially extended position when said first sheath catch-mechanism adjoins said third handle catch-mechanism; and
wherein said fourth handle catch-mechanism is adapted to prevent said sheath from retracting into said handle channel in the distal direction when said second sheath catch-mechanism adjoins said fourth handle-catch mechanism.

5. A kit comprising:
a coupler comprising a rigid encasement with an axial channel that releasably encloses the shaft of a trocar;
wherein the inner diameter of said channel substantially matches the outer diameter of said trocar shaft; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient; and
wherein the inner surface of the proximal end of said handle is formed with a first handle catch-mechanism that is adapted to interact with said coupler catch-mechanism;
a pathway within said handle channel in the distal direction is adapted to direct a trocar in a substantially divergent direction relative to said handle channel;
wherein said pathway is adapted to permit said trocar to slide backwards into a substantially retracted position within the handle and to be stopped in the retracted position when the distal end of said trocar has substantially reached the position of said divergent pathway in the distal direction, such that further travel by said trocar in the reverse (distal) direction is substantially blocked by the divergent direction of said divergent pathway in the distal direction;
wherein said pathway is adapted to permit said trocar to slide forwards and out of said handle when extracting said trocar out of said handle in the proximal direction; and
wherein said pathway is adapted to permit said coupler to slide forwards and out of said handle when extracting said trocar out of said handle in the proximal direction until said coupler catch-mechanism adjoins the first handle-catch mechanism at which point said first handle catch-mechanism is adapted to prevent said coupler from further sliding in the proximal direction out of said handle.

6. A kit according to claim 5, further comprising:
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a second handle catch-mechanism that is adapted to interact with said first sheath catch-mechanism;
wherein the transverse surface at the proximal end of said handle is formed with a third handle catch-mechanism that is adapted to interact with said second sheath catch-mechanism;
wherein said second handle catch-mechanism is adapted to releasably stop said sheath in a substantially extended position when said first sheath catch-mechanism adjoins said second handle catch-mechanism; and
wherein said third handle catch-mechanism is adapted to prevent said sheath from retracting into said handle channel in the distal direction when said second sheath catch-mechanism adjoins said third handle-catch mechanism.

7. A kit according to any one of claim 1, 2, 4, or 6;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism; and
wherein said sheath catch-release mechanism is adapted to release when sufficient pressure is exerted on the sheath catch-release mechanism so that the sheath becomes unlocked and may retract and slide into the handle in the backwards (distal) direction.

8. A kit according to any one of claim 1, 2, 4, or 6;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism;
wherein said sheath catch-release mechanism is adapted to release when sufficient pressure is exerted on the sheath catch-release mechanism so that the sheath becomes unlocked and may retract and slide into the handle in the backwards (distal) direction; and
wherein a position on the outer surface of said handle adjacent to said handle catch-release mechanism is formed to have a trigger-guard.

9. A sheath comprising:
a rigid encasement with an axial sheath channel that can enclose the shaft of a trocar;
wherein the inner diameter of said sheath channel substantially matches the outer diameter of said trocar, permitting said trocar to slide forwards or backwards within said sheath channel; and
wherein the length of said sheath covers the sharp tip of said trocar and a substantial length of the trocar's length.

10. A handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said aperture of said handle grip;
wherein said locking element comprises a rigid plate and a spring;
wherein said plate is formed with an axial locking element aperture through which said trocar is inserted;
wherein the diameter of said locking element aperture substantially matches the outer diameter of said trocar, permitting said trocar to slide forwards or backwards within said locking element aperture;
wherein said locking element aperture substantially aligns with said aperture of said handle grip;
wherein said spring is positioned behind said plate;
wherein said plate is adapted to form a substantially perpendicular angle, measured relative to the axial channel in the direction towards the proximal end of the handle grip, which permits the insertion of said trocar with a substantially small application of pushing force in the direction of insertion (the distal direction); and
wherein said plate is adapted to form an acute angle, measured relative to the axial channel in the direction towards the proximal end of the handle grip, which substantially resists and prevents the removal of said trocar in the direction of extraction (the proximal direction) due to said spring's compression force acting on the plate in the same direction as the direction of extraction.

11. A handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said handle grip aperture;
wherein said locking element comprises a rigid plate formed with an axial locking element aperture through which said trocar is inserted;
wherein the diameter of said locking element aperture substantially matches the outer diameter of said trocar, permitting said trocar to slide forwards or backwards within said locking element aperture;
wherein said locking element aperture substantially aligns with said handle grip aperture; and
wherein said locking element aperture is formed with protuberances adapted to permit the insertion of said trocar with a substantially small application of pushing force in the direction of insertion (the distal direction), but to resist with substantially large frictional pressure on the shaft of said trocar, which substantially restricts and prevents the removal of said trocar in the direction of extraction (the proximal direction).

12. A handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said handle grip aperture, comprising a rigid locking plate and locking rollers;
wherein said plate is formed with an axial locking element aperture through which said trocar is inserted;
wherein said plate comprises clips to hold said locking rollers in substantial alignment with said locking element aperture;
wherein the diameter of said locking element aperture substantially matches the outer diameter of said trocar, permitting said trocar to slide forwards or backwards within said locking element aperture;
wherein said locking element aperture substantially aligns with said handle grip aperture; and
wherein said locking rollers are formed with a substantially rough gripping surface, adapted to permit the insertion of said trocar with a substantially small application of pushing force in the direction of insertion (the distal direction), but to resist with substantially large frictional pressure on the shaft of said trocar, which substantially restricts and prevents the removal of said trocar in the direction of extraction (the proximal direction).

13. A device according to any one of claims 10-12, further comprising:
a rigid shield that is attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip; and
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip.

14. A device according to any one of claims 10-12, further comprising:
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip; and
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip.

15. A device according to any one of claims 10-12, further comprising:
a rigid shield that is attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim.

16. A device according to any one of claims 10-12, further comprising:
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim.

17. A method of protecting surgical personnel from sharps injuries while deploying a trocar comprising:
providing a kit comprising:
a coupler comprising a rigid encasement with an axial channel that releasably encloses the shaft of a trocar;
wherein the inner diameter of said channel substantially matches the outer diameter of said trocar shaft;
wherein said coupler is formed with feet adapted to exert inwards pressure and grip adjoining geometries formed in the shaft of a trocar when said trocar is pushed in the backwards direction into the coupler, and adapted to permit said trocar to slide in the forward direction through and out of the coupler, thereby releasably securing a trocar to said coupler in the backwards direction; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient;
wherein the inner surface of the distal end of said handle is formed with a first handle catch-mechanism that is adapted to interact with said coupler catch-mechanism;
wherein the inner surface of the proximal end of said handle is formed with a second handle catch-mechanism that is adapted to interact with said coupler catch-mechanism;
wherein said handle channel is adapted to permit said coupler to slide backwards into a substantially retracted position within said handle and adapted to releasably stop in the retracted position when said first handle catch-mechanism adjoins said coupler catch-mechanism and adapted to prevent said coupler and a trocar from retracting further beyond said first handle catch-mechanism into the handle in the reverse (distal) direction; and
wherein said handle channel is adapted to permit said coupler to slide forwards while not impeding the forward direction of said coupler and trocar when extracting said coupler and trocar out of said handle in the proximal direction, until said coupler catch-mechanism adjoins the second handle-catch mechanism at which point said second handle catch-mechanism is adapted to prevent said coupler from further sliding in the proximal direction out of said handle;
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a third handle catch-mechanism that is adapted to interact with said first sheath catch-mechanism;
wherein the transverse surface at the proximal end of said handle is formed with a fourth handle catch-mechanism that is adapted to interact with said second sheath catch-mechanism;
wherein said handle channel is adapted to stop said sheath in a substantially extended position when said first sheath catch-mechanism adjoins said third handle catch-mechanism;
wherein said fourth handle catch-mechanism is adapted to prevent said sheath from retracting into said handle channel in the distal direction when said second sheath catch-mechanism adjoins said fourth handle-catch mechanism;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism;
wherein said sheath catch-release mechanism is adapted to release when sufficient pressure is exerted on the sheath catch-release mechanism so that the sheath becomes unlocked and may retract and slide into the handle in the backwards (distal) direction; and
wherein a position on the outer surface of said handle adjacent to said handle catch-release mechanism is formed to have a trigger-guard;
a handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said aperture of said handle grip;
wherein said locking element comprises a rigid plate and a spring;
wherein said plate is formed with an axial locking element aperture through which said trocar is inserted;
wherein the diameter of said locking element aperture substantially matches the outer diameter of said trocar, permitting said trocar to slide forwards or backwards within said locking element aperture;
wherein said locking element aperture substantially aligns with said aperture of said handle grip;
wherein said spring is positioned behind said plate;
wherein said plate is adapted to form a substantially perpendicular angle, measured relative to the axial channel in the direction towards the proximal end of the handle grip, which permits the insertion of said trocar with a substantially small application of pushing force in the direction of insertion (the distal direction); and
wherein said plate is adapted to form an acute angle, measured relative to the axial channel in the direction towards the proximal end of the handle grip, which substantially resists and prevents the removal of said trocar in the direction of extraction (the proximal direction) due to said spring's compression force acting on the plate in the same direction as the direction of extraction;
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim;
applying said handle grip against the outer skin of a surgical site;
directing said sheath within the lumen towards and against said handle grip;
unlocking said sheath by depressing said sheath catch-release mechanism;
pushing a trocar and adjoining drainage tubing through the inner wall of said lumen into said handle grip;
withdrawing said trocar from said surgical site by withdrawing the handle grip into which the trocar has been locked; and
extricating said handle from the distal end of said drainage tubing and separating said shield from said handle grip for disposal of said handle, shield and handle grip into appropriate waste receptacles.

18. A method of protecting surgical personnel from sharps injuries while deploying a trocar comprising:
providing a kit comprising:
a coupler comprising a rigid encasement with an axial channel that releasably encloses the shaft of a trocar;
wherein the inner diameter of said channel substantially matches the outer diameter of said trocar shaft;
wherein said coupler is formed with feet adapted to exert inwards pressure and grip adjoining geometries formed in the shaft of a trocar when said trocar is pushed in the backwards direction into the coupler, and adapted to permit said trocar to slide in the forward direction through and out of the coupler, thereby releasably securing a trocar to said coupler in the backwards direction; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient;
wherein the inner surface of the distal end of said handle is formed with a first handle catch-mechanism that is adapted to interact with said coupler catch-mechanism;
wherein the inner surface of the proximal end of said handle is formed with a second handle catch-mechanism that is adapted to interact with said coupler catch-mechanism;
wherein said handle channel is adapted to permit said coupler to slide backwards into a substantially retracted position within said handle and adapted to releasably stop in the retracted position when said first handle catch-mechanism adjoins said coupler catch-mechanism and adapted to prevent said coupler and a trocar from retracting further beyond said first handle catch-mechanism into the handle in the reverse (distal) direction; and
wherein said handle channel is adapted to permit said coupler to slide forwards while not impeding the forward direction of said coupler and trocar when extracting said coupler and trocar out of said handle in the proximal direction, until said coupler catch-mechanism adjoins the second handle-catch mechanism at which point said second handle catch-mechanism is adapted to prevent said coupler from further sliding in the proximal direction out of said handle;
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a third handle catch-mechanism that is adapted to interact with said first sheath catch-mechanism;
wherein the transverse surface at the proximal end of said handle is formed with a fourth handle catch-mechanism that is adapted to interact with said second sheath catch-mechanism;
wherein said handle channel is adapted to stop said sheath in a substantially extended position when said first sheath catch-mechanism adjoins said third handle catch-mechanism; wherein said fourth handle catch-mechanism is adapted to prevent said sheath from retracting into said handle channel in the distal direction when said second sheath catch-mechanism adjoins said fourth handle-catch mechanism;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism;
wherein said sheath catch-release mechanism is adapted to release when sufficient pressure is exerted on the sheath catch-release mechanism so that the sheath becomes unlocked and may retract and slide into the handle in the backwards (distal) direction; and
wherein a position on the outer surface of said handle adjacent to said handle catch-release mechanism is formed to have a trigger-guard;
a handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said handle grip aperture;
wherein said locking element comprises a rigid plate formed with an axial locking element aperture through which said trocar is inserted;
wherein the diameter of said locking element aperture substantially matches the outer diameter of said trocar, permitting said trocar to slide forwards or backwards within said locking element aperture;
wherein said locking element aperture substantially aligns with said handle grip aperture; and
wherein said locking element aperture is formed with protuberances adapted to permit the insertion of said trocar with a substantially small application of pushing force in the direction of insertion (the distal direction), but to resist with substantially large frictional pressure on the shaft of said trocar, which substantially restricts and prevents the removal of said trocar in the direction of extraction (the proximal direction);
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim;
applying said handle grip against the outer skin of a surgical site;
directing said sheath within the lumen towards and against said handle grip;
unlocking said sheath by depressing said sheath catch-release mechanism;
pushing a trocar and adjoining drainage tubing through the inner wall of said lumen into said handle grip;
withdrawing said trocar from said surgical site by withdrawing the handle grip into which the trocar has been locked; and
extricating said handle from the distal end of said drainage tubing and separating said shield from said handle grip for disposal of said handle, shield and handle grip into appropriate waste receptacles.

19. A method of protecting surgical personnel from sharps injuries while deploying a trocar comprising:
providing a kit comprising:
a coupler comprising a rigid encasement with an axial channel that releasably encloses the shaft of a trocar;
wherein the inner diameter of said channel substantially matches the outer diameter of said trocar shaft;
wherein said coupler is formed with feet adapted to exert inwards pressure and grip adjoining geometries formed in the shaft of a trocar when said trocar is pushed in the backwards direction into the coupler, and adapted to permit said trocar to slide in the forward direction through and out of the coupler, thereby releasably securing a trocar to said coupler in the backwards direction; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient;
wherein the inner surface of the distal end of said handle is formed with a first handle catch-mechanism that is adapted to interact with said coupler catch-mechanism;
wherein the inner surface of the proximal end of said handle is formed with a second handle catch-mechanism that is adapted to interact with said coupler catch-mechanism;
wherein said handle channel is adapted to permit said coupler to slide backwards into a substantially retracted position within said handle and adapted to releasably stop in the retracted position when said first handle catch-mechanism adjoins said coupler catch-mechanism and adapted to prevent said coupler and a trocar from retracting further beyond said first handle catch-mechanism into the handle in the reverse (distal) direction; and
wherein said handle channel is adapted to permit said coupler to slide forwards while not impeding the forward direction of said coupler and trocar when extracting said coupler and trocar out of said handle in the proximal direction, until said coupler catch-mechanism adjoins the second handle-catch mechanism at which point said second handle catch-mechanism is adapted to prevent said coupler from further sliding in the proximal direction out of said handle;
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a third handle catch-mechanism that is adapted to interact with said first sheath catch-mechanism;
wherein the transverse surface at the proximal end of said handle is formed with a fourth handle catch-mechanism that is adapted to interact with said second sheath catch-mechanism;
wherein said handle channel is adapted to stop said sheath in a substantially extended position when said first sheath catch-mechanism adjoins said third handle catch-mechanism;
wherein said fourth handle catch-mechanism is adapted to prevent said sheath from retracting into said handle channel in the distal direction when said second sheath catch-mechanism adjoins said fourth handle-catch mechanism;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism;
wherein said sheath catch-release mechanism is adapted to release when sufficient pressure is exerted on the sheath catch-release mechanism so that the sheath becomes unlocked and may retract and slide into the handle in the backwards (distal) direction; and
wherein a position on the outer surface of said handle adjacent to said handle catch-release mechanism is formed to have a trigger-guard;
a handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said handle grip aperture, comprising a rigid locking plate and locking rollers;
wherein said plate is formed with an axial locking element aperture through which said trocar is inserted;
wherein said plate comprises clips to hold said locking rollers in substantial alignment with said locking element aperture;
wherein the diameter of said locking element aperture substantially matches the outer diameter of said trocar, permitting said trocar to slide forwards or backwards within said locking element aperture;
wherein said locking element aperture substantially aligns with said handle grip aperture; and
wherein said locking rollers are formed with a substantially rough gripping surface, adapted to permit the insertion of said trocar with a substantially small application of pushing force in the direction of insertion (the distal direction), but to resist with substantially large frictional pressure on the shaft of said trocar, which substantially restricts and prevents the removal of said trocar in the direction of extraction (the proximal direction);
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim;
applying said handle grip against the outer skin of a surgical site;
directing said sheath within the lumen towards and against said handle grip;
unlocking said sheath by depressing said sheath catch-release mechanism;
pushing a trocar and adjoining drainage tubing through the inner wall of said lumen into said handle grip;
withdrawing said trocar from said surgical site by withdrawing the handle grip into which the trocar has been locked; and
extricating said handle from the distal end of said drainage tubing and separating said shield from said handle grip for disposal of said handle, shield and handle grip into appropriate waste receptacles.

20. A method of protecting surgical personnel from sharps injuries while deploying a trocar comprising:
providing a kit comprising:
a coupler comprising a rigid encasement with an axial channel that releasably encloses the shaft of a trocar;
wherein the inner diameter of said channel substantially matches the outer diameter of said trocar shaft; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient; and
wherein the inner surface of the proximal end of said handle is formed with a first handle catch-mechanism that is adapted to interact with said coupler catch-mechanism;
a pathway within said handle channel in the distal direction is adapted to direct a trocar in a substantially divergent direction relative to said handle channel;
wherein said pathway is adapted to permit said trocar to slide backwards into a substantially retracted position within the handle and to be stopped in the retracted position when the distal end of said trocar has substantially reached the position of said divergent pathway in the distal direction, such that further travel by said trocar in the reverse (distal) direction is substantially blocked by the divergent direction of said divergent pathway in the distal direction;
wherein said pathway is adapted to permit said trocar to slide forwards and out of said handle when extracting said trocar out of said handle in the proximal direction; and
wherein said pathway is adapted to permit said coupler to slide forwards and out of said handle when extracting said trocar out of said handle in the proximal direction until said coupler catch-mechanism adjoins the first handle-catch mechanism at which point said first handle catch-mechanism is adapted to prevent said coupler from further sliding in the proximal direction out of said handle;
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a second handle catch-mechanism that is adapted to interact with said first sheath catch-mechanism;
wherein the transverse surface at the proximal end of said handle is formed with a third handle catch-mechanism that is adapted to interact with said second sheath catch-mechanism;
wherein said second handle catch-mechanism is adapted to releasably stop said sheath in a substantially extended position when said first sheath catch-mechanism adjoins said second handle catch-mechanism;
wherein said third handle catch-mechanism is adapted to prevent said sheath from retracting into said handle channel in the distal direction when said second sheath catch-mechanism adjoins said third handle-catch mechanism;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism;
wherein said sheath catch-release mechanism is adapted to release when sufficient pressure is exerted on the sheath catch-release mechanism so that the sheath becomes unlocked and may retract and slide into the handle in the backwards (distal) direction; and
wherein a position on the outer surface of said handle adjacent to said handle catch-release mechanism is formed to have a trigger-guard;
a handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said aperture of said handle grip;
wherein said locking element comprising a rigid plate and a spring;
wherein said plate is formed with an axial locking element aperture through which said trocar is inserted;
wherein the diameter of said locking element aperture substantially matches the outer diameter of said trocar, permitting said trocar to slide forwards or backwards within said locking element aperture;
wherein said locking element aperture substantially aligns with said aperture of said handle grip;
wherein said spring is positioned behind said plate;
wherein said plate is adapted to form a substantially perpendicular angle, measured relative to the axial channel in the direction towards the proximal end of the handle grip, which permits the insertion of said trocar with a substantially small application of pushing force in the direction of insertion (the distal direction); and
wherein said plate is adapted to form an acute angle, measured relative to the axial channel in the direction towards the proximal end of the handle grip, which substantially resists and prevents the removal of said trocar in the direction of extraction (the proximal direction) due to said spring's compression force acting on the plate in the same direction as the direction of extraction;
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim;
applying said handle grip against the outer skin of a surgical site;
directing said sheath within the lumen towards and against said handle grip;
unlocking said sheath by depressing said sheath catch-release mechanism;
pushing a trocar and adjoining drainage tubing through the inner wall of said lumen into said handle grip;
withdrawing said trocar from said surgical site by withdrawing the handle grip into which the trocar has been locked; and
extricating said handle from the distal end of said drainage tubing and separating said shield from said handle grip for disposal of said handle, shield and handle grip into appropriate waste receptacles.

21. A method of protecting surgical personnel from sharps injuries while deploying a trocar comprising:
providing a kit comprising:
a coupler comprising a rigid encasement with an axial channel that releasably encloses the shaft of a trocar;
wherein the inner diameter of said channel substantially matches the outer diameter of said trocar shaft; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient; and
wherein the inner surface of the proximal end of said handle is formed with a first handle catch-mechanism that is adapted to interact with said coupler catch-mechanism;
a pathway within said handle channel in the distal direction is adapted to direct a trocar in a substantially divergent direction relative to said handle channel;
wherein said pathway is adapted to permit said trocar to slide backwards into a substantially retracted position within the handle and to be stopped in the retracted position when the distal end of said trocar has substantially reached the position of said divergent pathway in the distal direction, such that further travel by said trocar in the reverse (distal) direction is substantially blocked by the divergent direction of said divergent pathway in the distal direction;
wherein said pathway is adapted to permit said trocar to slide forwards and out of said handle when extracting said trocar out of said handle in the proximal direction; and
wherein said pathway is adapted to permit said coupler to slide forwards and out of said handle when extracting said trocar out of said handle in the proximal direction until said coupler catch-mechanism adjoins the first handle-catch mechanism at which point said first handle catch-mechanism is adapted to prevent said coupler from further sliding in the proximal direction out of said handle;
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a second handle catch-mechanism that is adapted to interact with said first sheath catch-mechanism;
wherein the transverse surface at the proximal end of said handle is formed with a third handle catch-mechanism that is adapted to interact with said second sheath catch-mechanism;
wherein said second handle catch-mechanism is adapted to releasably stop said sheath in a substantially extended position when said first sheath catch-mechanism adjoins said second handle catch-mechanism;
wherein said third handle catch-mechanism is adapted to prevent said sheath from retracting into said handle channel in the distal direction when said second sheath catch-mechanism adjoins said third handle-catch mechanism;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism;
wherein said sheath catch-release mechanism is adapted to release when sufficient pressure is exerted on the sheath catch-release mechanism so that the sheath becomes unlocked and may retract and slide into the handle in the backwards (distal) direction; and
wherein a position on the outer surface of said handle adjacent to said handle catch-release mechanism is formed to have a trigger-guard;
a handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said handle grip aperture;
wherein said locking element comprises a rigid plate formed with an axial locking element aperture through which said trocar is inserted;
wherein the diameter of said locking element aperture substantially matches the outer diameter of said trocar, permitting said trocar to slide forwards or backwards within said locking element aperture;
wherein said locking element aperture substantially aligns with said handle grip aperture; and
wherein said locking element aperture is formed with protuberances adapted to permit the insertion of said trocar with a substantially small application of pushing force in the direction of insertion (the distal direction), but to resist with substantially large frictional pressure on the shaft of said trocar, which substantially restricts and prevents the removal of said trocar in the direction of extraction (the proximal direction);
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim;
applying said handle grip against the outer skin of a surgical site;
directing said sheath within the lumen towards and against said handle grip;
unlocking said sheath by depressing said sheath catch-release mechanism;
pushing a trocar and adjoining drainage tubing through the inner wall of said lumen into said handle grip;
withdrawing said trocar from said surgical site by withdrawing the handle grip into which the trocar has been locked; and
extricating said handle from the distal end of said drainage tubing and separating said shield from said handle grip for disposal of said handle, shield and handle grip into appropriate waste receptacles.

22. A method of protecting surgical personnel from sharps injuries while deploying a trocar comprising:
providing a kit comprising:
a coupler comprising a rigid encasement with an axial channel that releasably encloses the shaft of a trocar;
wherein the inner diameter of said channel substantially matches the outer diameter of said trocar shaft; and
wherein the distal end of said coupler is formed with a coupler catch-mechanism on the outer surface of said coupler;
a handle comprising a rigid encasement with an axial handle channel that releasably encloses said coupler and the shaft of a trocar;
wherein the inner diameter of said handle channel substantially matches the outer diameter of said coupler, permitting said coupler to slide forwards or backwards within said handle channel;
wherein the outside diameter of said handle is formed to substantially fit within the hand of a clinician holding the handle;
wherein the transverse edge of said handle at the proximal end is formed with a surface that extends outwards in the transverse direction from said handle channel with an area integrated from the outer diameter of said handle channel to an outer diameter of sufficient width to provide substantial physical support to cover the clinician's hand when the clinician pushes the proximal end of said handle into the skin of a patient; and
wherein the inner surface of the proximal end of said handle is formed with a first handle catch-mechanism that is adapted to interact with said coupler catch-mechanism;
a pathway within said handle channel in the distal direction is adapted to direct a trocar in a substantially divergent direction relative to said handle channel;
wherein said pathway is adapted to permit said trocar to slide backwards into a substantially retracted position within the handle and to be stopped in the retracted position when the distal end of said trocar has substantially reached the position of said divergent pathway in the distal direction, such that further travel by said trocar in the reverse (distal) direction is substantially blocked by the divergent direction of said divergent pathway in the distal direction;
wherein said pathway is adapted to permit said trocar to slide forwards and out of said handle when extracting said trocar out of said handle in the proximal direction; and
wherein said pathway is adapted to permit said coupler to slide forwards and out of said handle when extracting said trocar out of said handle in the proximal direction until said coupler catch-mechanism adjoins the first handle-catch mechanism at which point said first handle catch-mechanism is adapted to prevent said coupler from further sliding in the proximal direction out of said handle;
a sheath comprising a rigid encasement with an axial sheath channel that releasably encloses the shaft of a trocar;
wherein the inner diameter of said sheath channel substantially matches the outer diameter of said trocar, permitting said trocar to slide forwards or backwards within said channel;
wherein the length of said sheath covers the sharp tip of said trocar and a substantial length of the trocar's length;
wherein the distal end of said sheath is formed with a first sheath catch-mechanism on the outer surface of said sheath;
wherein the proximal end of said sheath is formed with a second sheath catch-mechanism on the outer surface of said sheath;
wherein the diameter of said sheath is adapted to fit within the inner diameter of said handle and outside the outer diameter of said coupler, permitting said sheath to slide forwards or backwards within said handle and permitting said coupler to slide forwards or backwards within said sheath;
wherein the inner surface of the proximal end of said handle is formed with a second handle catch-mechanism that is adapted to interact with said first sheath catch-mechanism;
wherein the transverse surface at the proximal end of said handle is formed with a third handle catch-mechanism that is adapted to interact with said second sheath catch-mechanism;
wherein said second handle catch-mechanism is adapted to releasably stop said sheath in a substantially extended position when said first sheath catch-mechanism adjoins said second handle catch-mechanism;
wherein said third handle catch-mechanism is adapted to prevent said sheath from retracting into said handle channel in the distal direction when said second sheath catch-mechanism adjoins said third handle-catch mechanism;
wherein the outer surface of said sheath at the distal end is formed with a sheath catch-release mechanism;
wherein the inner surface of said handle at the proximal end is formed with a handle catch-release mechanism adapted to interact with said sheath catch-release mechanism;
wherein the interaction of said sheath catch-release mechanism with said handle catch-release mechanism is adapted to releasably lock the sheath in a position extending outwards from the handle and adapted to prevent retraction of said sheath into the handle in the distal direction beyond said handle catch-release mechanism;
wherein said sheath catch-release mechanism is adapted to release when sufficient pressure is exerted on the sheath catch-release mechanism so that the sheath becomes unlocked and may retract and slide into the handle in the backwards (distal) direction; and
wherein a position on the outer surface of said handle adjacent to said handle catch-release mechanism is formed to have a trigger-guard;
a handle grip comprising:
a rigid encasement with an axial handle grip channel that encloses the shaft of a trocar;
wherein the inner diameter of said handle grip channel substantially matches the outer diameter of a trocar, permitting said trocar to slide forwards or backwards within said handle grip channel;
wherein said handle grip is formed with an axial handle grip aperture at the proximal end of said handle grip through which a trocar is inserted;
wherein the diameter of said handle grip aperture substantially matches the outer diameter of a trocar, permitting said trocar to slide forwards or backwards within said handle grip aperture;
wherein the outer diameter of said handle grip is formed to substantially fit within the hand of a clinician holding the handle grip; and
wherein said handle grip is formed with a backstop for a trocar at the distal end;
a locking element mounted inside said handle grip positioned at the proximal end of said handle grip and positioned behind said handle grip aperture, comprising a rigid locking plate and locking rollers;
wherein said plate is formed with an axial locking element aperture through which said trocar is inserted;
wherein said plate comprises clips to hold said locking rollers in substantial alignment with said locking element aperture;
wherein the diameter of said locking element aperture substantially matches the outer diameter of said trocar, permitting said trocar to slide forwards or backwards within said locking element aperture;
wherein said locking element aperture substantially aligns with said handle grip aperture; and
wherein said locking rollers are formed with a substantially rough gripping surface, adapted to permit the insertion of said trocar with a substantially small application of pushing force in the direction of insertion (the distal direction), but to resist with substantially large frictional pressure on the shaft of said trocar, which substantially restricts and prevents the removal of said trocar in the direction of extraction (the proximal direction);
a rigid shield that is removably attached to the proximal end of said handle grip;
wherein said shield is formed with an axial shield aperture and axial shield channel, through which a trocar is passed;
wherein the diameter of said shield aperture and inner diameter of said shield channel substantially match the outer diameter of said trocar, permitting said trocar to slide forwards or backwards through said shield aperture and within said shield channel;
wherein said shield aperture substantially aligns with said aperture of said handle grip;
wherein the transverse edge of said shield at the proximal end is formed with a surface that extends outwards in the transverse direction from said shield aperture with an area integrated from the outer diameter of said shield to an outer diameter that substantially covers a clinician's hand when holding said handle grip; and
wherein said shield is formed with a raised rim along the outer circumference of said shield, which rises in the proximal direction by a height that substantially prevents the tip of a trocar from sliding beyond the rim;
applying said handle grip against the outer skin of a surgical site;
directing said sheath within the lumen towards and against said handle grip;
unlocking said sheath by depressing said sheath catch-release mechanism;
pushing a trocar and adjoining drainage tubing through the inner wall of said lumen into said handle grip;
withdrawing said trocar from said surgical site by withdrawing the handle grip into which the trocar has been locked; and
extricating said handle from the distal end of said drainage tubing and separating said shield from said handle grip for disposal of said handle, shield and handle grip into appropriate waste receptacles.
